(19) United States
(12) Patent Application Publication
(10) Pub. No.: US 2023/0210888 A1
(43) Pub. Date: Jul. 6, 2023

(54) PHOTOTHERANOSTIC NANOAGENTS WITH EXCELLENT ATHEROSCLEROTIC PLAQUE-TARGETING AND PLAQUE-PENETRATING PROPERTIES, AND USE THEREOF

(71) Applicants: Korea University Research and Business Foundation, Seoul (KR); Chung Ang University Industry Academic Cooperation Foundation, Seoul (KR); KOREA ADVANCED INSTITUTE OF SCIENCE AND TECHNOLOGY, Daejeon (KR)

(72) Inventors: Kyeongsoon PARK, Gangwon-do (KR); Jin Won KIM, Seoul (KR); Hongki YOO, Daejeon (KR); Jin Hyuk KIM, Incheon (KR)

(21) Appl. No.: 18/062,806

(22) Filed: Dec. 7, 2022

(30) Foreign Application Priority Data
Dec. 31, 2021 (KR) 10-2021-0193912

(51) Int. Cl. A61K 31/716; A61K 49/00; A61P 9/10

(52) U.S. Cl. CPC A61K 31/716; A61K 49/0017; A61P 9/10; B82Y 5/00

(57) ABSTRACT

The present disclosure provides nanoparticles including laminarin and a near-infrared responsive photosensitizer covalently bonded thereto, and a composition for preventing, diagnosing, or treating arteriosclerosis comprising the same as an active ingredient. According to the present disclosure, not only the atherosclerotic plaque targeting and plaque-penetrating properties can be enhanced as compared to conventional photodynamic therapy, thus capable of being usefully used for in vivo imaging of atherosclerotic plaques, but also the size of atherosclerotic plaques is reduced by inducing apoptosis of macrophages in atherosclerotic plaques, thus capable of stabilizing atherosclerotic plaques. Therefore, the composition comprising the nanoparticles of the present disclosure as an active ingredient is expected to be usefully used for the prevention, diagnosis and/or treatment of arteriosclerosis, in that photodynamic therapy and image diagnostic of arteriosclerosis can be performed simultaneously or sequentially.

### CROSS-REFERENCE TO RELATED APPLICATIONS

This application claims the benefit and priority to Korean Patent Application No. 10-2021-0193912, filed on Dec. 31, 2021. The entire disclosure of the application identified in this paragraph is incorporated herein by reference.

### TECHNICAL FIELD

The present disclosure has been made with the support of the Ministry of Science and ICT of the Republic of Korea, under Project Specific Number 1711126956 and Detailed Project Number 2019M3A9E2066882, wherein the research management institution for the above project is the National Research Foundation of Korea, the research project name is “Bio-Medical Technology Development (R&D)”, the research task name is “In vivo evaluation of high-risk arteriosclerotic plaque image diagnostic and phototherapy for intra-coronary rupture”, the project performing institution is Korea University, and the research period is 2021.01.01-2021.12.31. Also, the present disclosure has been made with the support of the Ministry of Science and ICT of the Republic of Korea, under Project Specific Number 1711126959 and Detailed Project Number 2019M3A9E2066883, wherein the research management institution for the above project is the National Research Foundation of Korea, the research project name is “Bio-Medical Technology Development (R&D)”, the research task name is “Development of fusion materials for targeted diagnosis and treatment of high-risk arteriosclerosis plaques”, the project performing institution is Chung-Ang University, and the research period is 2021.01.01-2021.12.31. Further, the present disclosure has been made with the support of the Ministry of Science and ICT of the Republic of Korea, under Project Specific Number 1711129021 and Detailed Project Number 2019M3A9E2066880, wherein the research management institution for the above project is the National Research Foundation of Korea, the research project name is “Bio-Medical Technology Development (R&D)”, the research project name is “Development of an intravascular optical tomography-molecular imaging-phototherapy fusion catheter system”, the project performing institution is Korea Advanced Institute of Science and Technology, and the research period is 2021.01.01-2021.12.31.

The present disclosure relates to a phototheranostic nanoagents for photodynamic therapy and image diagnosis with excellent atherosclerotic plaque-targeting and plaque-penetrating properties.

### BACKGROUND

Atherosclerosis is a disease in which cholesterol, etc. are deposited on the arterial wall, the blood vessel gradually narrows or clogs, and the arterial endothelial cells proliferate, causing the arterial wall to thicken, harden, and causing loss of elasticity, resulting in blood flow disturbance.

The pathogenesis of atherosclerosis begins with damage to arterial endothelial cells due to high blood pressure, high blood cholesterol, smoking, high blood sugar, and the like. Low density lipoprotein (LDL), which is a carrier of blood cholesterol, enters damaged arterial endothelial cells, accumulates in the tunica intima, and is oxidized. Damaged endothelial cells express adhesion molecules on the cell surface, and monocytes adhere to the adhesion molecules and invade into the tunica intima. The cells are stimulated by oxidized LDL, and mononucleosis differentiates into macrophages by M-CSF (monocyte-colony stimulating factor) secreted by endothelial cells. Macrophage absorbs oxidized LDL through a scavenger receptor, and its ability to regulate intracellular cholesterol concentration is impaired, resulting in foam cells in which cholesterol is accumulated at high concentrations.

Foam cell forms a fatty streak, which is an early lesion of atherosclerosis. As the lesion progresses further, smooth muscle cells (SMC) of the tunica media enter the tunica intima and proliferate or become foam cells, secreting extracellular matrix to form a fibrous cap. In the lesion, calcium, cholesterol, lipid, collagen, macrophage, foam cell, cell debris, smooth muscle cell, and the like are gradually accumulated to form an atherosclerotic plaque.

Atherosclerotic plaque is relatively stable under the endothelium until the endothelium directive above is damaged. However, necrosis and apoptosis of macrophages and smooth muscle cells, secretion of cytomatrix-degrading enzymes by macrophages, etc., may cause instability and rupture. In addition, the damaged endothelium can no longer produce blood clotting inhibitors, and thus, a clot of blood cells attached to the blood vessel wall forms a thrombus. Atherosclerotic plaque rupture and thrombotic complications lead to fatal acute coronary syndrome, resulting in a leading cause of death worldwide.

As mentioned above, macrophage that significantly contributes to the pathogenesis of atherosclerosis expresses Dectin-1. Dectin-1 is a protein encoded by the human CLEC7A gene and is one of the C-type lectin/C-type lectin-like domain (CTL/CTLD) superfamily. Its cytoplasmic domain has a partial immunoreceptor tyrosine-based activation motif and is known to contribute to the innate immune response.

In addition, Dectin-1 functions as a pattern-recognition receptor for glucans linked by β(1→3) and β(1→6) bonds from fungi and plants. Dectin-1 ligands include β-glucan and laminarin. In particular, Dectin-1 is known to be expressed in macrophages of mouse atherosclerotic plaques (Brown GD et al. J Exp Med. 2002; and Szilagyi K et al. Atherosclerosis, 2015).

Currently, a method for inhibiting or preventing disease progression and improving blood circulation in narrowed blood vessels is used for the treatment of arteriosclerosis. Drug therapy for inhibiting or preventing disease progression includes the administration of aspirin to prevent blood clots, or the administration of lipid-lowering drugs such as statins to lower blood cholesterol levels. Therapies for improving blood circulation in narrowed blood vessels include angioplasty, such as balloon angioplasty or stenting, and bypass grafting, which is a surgical procedure. However, there is no therapeutic method capable of stabilizing or reducing the arteriosclerosis that has already occurred.

Recently, in non-clinical studies, therapies capable of simultaneously performing photoactivatable therapy and diagnosis are being investigated for the treatment of arteriosclerosis. Therefore, if a phototheranostic fusion material that exhibits the stabilization effect of atherosclerotic plaques capable of simultaneously performing photoactivatable therapy and diagnostic can be developed, it is expected that a non-invasive fundamental therapy with high selectivity and specificity compared to conventional prophylactic and surgical therapy is possible.

### PRIOR ART DOCUMENTS

### Patent Document

KR 10-2021-0091508 A (published on Jul. 22, 2021)

### SUMMARY

### Technical Problem

The present inventors have made intensive researches to develop a phototheranostic fusion material that exhibits atherosclerotic plaque stabilization effect. As a result, the inventors have developed novel nanoparticles comprising a near-infrared responsive photosensitizer covalently bound to laminarin. In addition, the inventors have found that as a result of irradiating a near-infrared light after administration of a composition containing the nanoparticles as an active ingredient, a near-infrared fluorescence (NIFR) signal is emitted from atherosclerotic plaque and at the same time, singlet oxygen (¹0₂) is generated, thereby being effective in providing in vivo images of atherosclerotic plaques, reducing the size of atherosclerotic plaques and reducing macrophage-mediated inflammatory responses. The present disclosure has been completed on the basis of such finding.

Therefore, it is an object of the present disclosure to provide nanoparticles comprising laminarin and a near-infrared responsive photosensitizer covalently bound thereto.

It is another object of the present disclosure to provide a composition for preventing, diagnosing, or treating arteriosclerosis comprising the above-mentioned nanoparticles as an active ingredient.

It is yet another object of the present disclosure to provide a method for preventing, diagnosing, or treating arteriosclerosis comprising administering to a subject in need of administering a composition comprising the above-mentioned nanoparticles as an active ingredient.

### Solution to Problem

The present inventors have made intensive research to develop a phototheranostic fusion material that exhibits atherosclerotic plaque stabilization effect. As a result, the inventors have developed novel nanoparticles comprising a near-infrared responsive photosensitizer covalently bound to laminarin. In addition, the inventors have found that as a result of irradiating a near-infrared light after administration of a composition containing it as an active ingredient, a near-infrared fluorescence signal is emitted from atherosclerotic plaque and at the same time, singlet oxygen is generated, thereby being effective in providing in vivo images of atherosclerotic plaques, reducing the size of atherosclerotic plaques and reducing macrophage-mediated inflammatory responses.

The present disclosure relates to a phototheranostic nanoparticle fusion material capable of simultaneously performing photodynamic therapy and image diagnosis for arteriosclerotic plaques.

Hereinafter, the present disclosure will be described in more detail.

According to one aspect of the present disclosure, there are provided nanoparticles comprising a conjugate of laminarin and a near-infrared responsive photosensitizer covalently bonded thereto.

As used herein, the term “laminarin” means a linear polysaccharide composed of β(1→3) glucan having β(1→6) branches. Laminarin is known to be a hypotonic glucan found in brown algae and to be produced by photosynthesis. Laminarin has a β(1→3):β(1→6) binding ratio of 3:1 and is water soluble. Laminarin can be obtained by separation and purification from brown algae, and is commercially available from Sigma-Aldrich, and the like.

Laminarin is a low molecular β-glucan known to be biologically inactive. Since the vertebrates do not have β(1→3) glucans, it is known that small molecule β-glucans such as laminarin can exist stably for a long period of time in mammalian systems.

In one embodiment of the present disclosure, the laminarin is a compound represented by the following Chemical Formula 1:

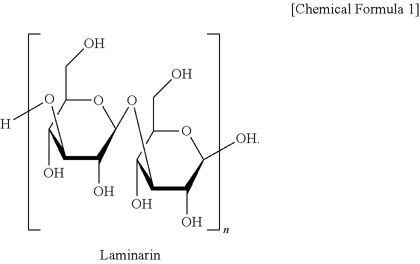

In one embodiment of the present disclosure, the average molecular weight of the laminarin is about 5 kDa. In another embodiment of the present disclosure, the average molecular weight of the laminarin is 1 kDa to 9 kDa, 2 kDa to 8 kDa, 3 kDa to 7 kDa, or 4 kDa to 6 kDa.

In one embodiment of the present disclosure, the laminarin is contained in an amount of 84 wt % to 86 wt % based on the total weight (wt %) of the nanoparticles. In another embodiment of the present disclosure, the laminarin is contained in an amount of 60 wt % to 95 wt %, 70 wt % to 95 wt %, 75 wt % to 95 wt %, 80 wt % to 95 wt %, 80 wt % to 90 wt %, 81 wt % to 89 wt %, 82 wt % to 88 wt %, 83 wt % to 87 wt %, or 84 wt % to 86 wt % based on the total weight of the nanoparticles, but is not limited thereto.

As used herein, the term “photosensitizer” means a substance that induces physicochemical changes in neighboring molecules by donating electrons to a substrate or extracting hydrogen atoms from the substrate. Generally, the photosensitizer absorbs electromagnetic radiation and transfers the absorption energy to neighboring molecules. This light absorption of the photosensitizer is enabled by a large delocalized π-system, which lowers the energy of the HOMO (Highest Occupied Molecular Orbital) and LUMO (Lowest Unoccupied Molecular) orbitals to promote photoexcitation.

The photosensitizer effective for use in photodynamic therapy (PDT) has the characteristics such as low dark toxicity, selective absorption to target tissues, short duration of skin photosensitivity, minimization of patient pain, non-mutagenic, suitable route of administration such as systemic administration; oral administration; topical administration, chemical stability, and water solubility to facilitate transport in the body.

As a PDT light source, a laser, a light emitting diode (LED), and a lamp are mainly used.

In one embodiment of the present disclosure, the near-infrared light source may be a laser, LED, or lamp, but is not necessarily limited thereto, and it can be used without limitation as long as it is a light source known in the art that can be used in the PDT.

In one embodiment of the present disclosure, the near-infrared responsive photosensitizer is contained in an amount of 5 wt % to 40 wt %, 5 wt % to 30 wt %. 5 wt % to 15 wt %, 10 wt % to 40 wt %, 10 wt % to 30 wt %, or 10 wt % to 20 wt % based on the total weight of nanoparticles. In another embodiment of the present disclosure, the near-infrared responsive photosensitizer is contained in an amount of 10 wt % to 20 wt %, 12 wt % to 18 wt %, or 14 wt % to 16 wt % based on the total weight of nanoparticles.

As used herein, the term “near infrared” means light having a wavelength in the range of 600 nm to 800 nm, but is not limited thereto, and may include a wavelength in the above range and a wavelength in the range of ±20%. When using light with a wavelength of less than 600 nm and a photosensitizer that responds thereto, it is absorbed by oxyhemoglobin and deoxyhemoglobin, and the selectivity to target tissues is reduced. When using light with a wavelength of more than 800 nm and a photosensitizer that responds thereto, light penetration is inhibited because of the characteristic that water absorbs light, which is inefficient in exciting oxygen molecules from a triplet ground state to a singlet excitation state.

As used herein, the term “near infrared responsive photosensitizer” means a substance that absorbs near-infrared as defined above and transfers the absorption energy to neighboring molecules to cause a physicochemical change.

In one embodiment of the present disclosure, the near-infrared responsive photosensitizer is at least one selected from the group consisting of chlorin e6 (Ce6), porphyrin, photofrin, temoporfin, aminolevulinic acid-induced protoporphyrin IX, motexafin lutetium, padoporfin, padeliporfin, talaporfin (NPe₆), radachlorin, Purlytin, phthalocyanines, Verteporfin, HPPH (photochlor), TPC (5-(4-carboxyphenyl)-10,15,20-triphenyl-2,3-dihydroxychlorin), Chlorin p6 (Cp6), Purpurin-18, purpurinimide, and bacteriochlorin, but is not necessarily limited thereto, and any photosensitizer capable of absorbing a near-infrared light among photosensitizers known in the art can be used without limitation.

In one embodiment of the present disclosure, the near-infrared responsive photosensitizer is chlorine e6.

As used herein, the term “Chlorin e6 (Ce6)” has a molecular formula of C₃₄H₃₆N₄O₆, and the IUPAC name means a photosensitizer which is (17S,18S)-18-(2-carboxyethyl)-20-(carboxymethyl)-12-ethenyl-7-ethyl-3,8,13,17-tetramethyl-17,18,22,23-tetrahydroporphyrin-2-carboxylic acid.

In one embodiment of the present disclosure, the Chlorine e6 is a compound represented by the following Chemical Formula 2:

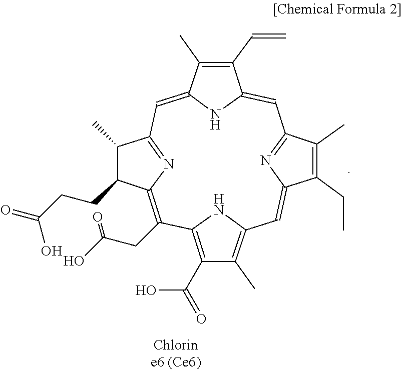 [SMILES: C=Cc1c(C)c2cc3nc(c(CC(=O)O)c4[nH]c(cc5nc(cc1[nH]2)C(C)=C5CC)c(C)c4C(=O)O)[C@@H](CCC(=O)O)[C@@H]3C]

In one embodiment of the present disclosure, the Chlorine e6 is contained in an amount of 14 wt % to 16 wt %, based on the total weight of the nanoparticles. In another embodiment of the present disclosure, the Chlorine e6 is contained in an amount of 10 wt % to 20 wt %, 12 wt % to 18 wt %, or 14 wt % to 16 wt %, based on the total weight of the nanoparticles.

As used herein, the term “covalent bond” means a chemical bond including electron pair sharing between certain atoms.

In one embodiment of the present disclosure, the covalent bond may be formed by further adding a crosslinking agent to the laminarin and the photosensitizer.

In one embodiment of the present disclosure, the crosslinking agent can be used without limitation as long as it is a carboxyl group activator and/or an esterification catalyst known in the art. For example, EDC (1-(3-Dimethylaminopropyl)-3-ethylcarbodiimide hydrochloride), DCC (dicyclohexyl carbodiimide), DIC (N,N′-Diisopropylcarbodiimide), NHS (N-hydroxysuccinimide), Sulfo-NHS (Nhydroxysulfosuccinimide), imidoester-based crosslinking agent, Sulfo-SMCC (sulfosuccinimidyl 4-(N-maleimidomethyl)cyclohexane-1-carboxylate), DMAP (4-Dimethylaminopyridine), or a combination thereof can be used, but is not necessarily limited thereto, and chemical crosslinking agents commonly used in the technical field to which the present disclosure belongs can be used without limitation.

As used herein, the term “photodynamic therapy (PDT)” means a dynamic interaction between a photosensitizer, oxygen and light. PDT initiates a dynamic interaction by treating light of a suitable photosensitizer-specific wavelength and activating the photosensitizer, and thus, the absorption wavelength of the photosensitizer and the wavelength of the light source should match with each other. Most photosensitizers have multiple absorption peaks, for example, a red Q-band in the range of 600 nm to 700 nm and a blue Soret band around 400 nm, but longer wavelength light sources can penetrate deeper into the tissue to treat deeper target tissues, and thus longer wavelength light sources are mainly used for PDT.

The optical penetration depth, i.e., the depth at which the light intensity decreases to 37% of its initial intensity in tissue, is wavelength-dependent and is 0.5 mm or less at a wavelength ranging from 400 nm to 430 nm, 1 mm at a wavelength of 500 nm, 2 nm to 3 mm at a wavelength of 630 nm, and 5 mm to 6 mm at a wavelength of 700 nm to 800 nm.

In one embodiment of the present disclosure, the laminarin is located outside the nanoparticles and the photosensitizer is located inside the nanoparticles.

In one embodiment of the present disclosure, the laminarin is hydrophilic, and the photosensitizer is hydrophobic.

In one embodiment of the present disclosure, the nanoparticles have a spherical morphology in which in aqueous solution, the hydrophilic laminarin is located outside the nanoparticles, and the hydrophobic photosensitizer is located inside the nanoparticles.

In one embodiment of the present disclosure, the nanoparticles are produced by the self-assembly of conjugates.

In one embodiment of the present disclosure, the nanoparticles are self-assembled in aqueous solution.

As used herein, the term “self-assembly” means a spontaneous assembly process in which smaller subunits form larger and well-organized patterns.

As used herein, the term “self-assembled nanoparticles” means nanoparticles that are spontaneously assembled as a result of interactions between particles aimed at achieving thermodynamic equilibrium and reducing the free energy of a system.

In one embodiment of the present disclosure, the hydrodynamic diameter of the nanoparticles as measured by a dynamic light scattering (DLS) method is 10 to 500 nm, 10 to 400 nm, 10 to 350 nm, 10 to 300 nm, 10 to 250 nm, 10 to 220 nm, 10 to 200 nm, 10 to 150 nm, 10 to 140 nm, 10 to 130 nm, 10 to 120 nm, or 10 to 110 nm; 50 to 500 nm, 50 to 400 nm, 50 to 350 nm, 50 to 300 nm, 50 to 250 nm, 50 to 220 nm, 50 to 200 nm, 50 to 150 nm, 50 to 140 nm, 50 to 130 nm, 50 to 120 nm, or 50 to 110 nm; 80 to 500 nm, 80 to 400 nm, 80 to 350 nm, 80 to 300 nm, 80 to 250 nm, 80 to 220 nm, 80 to 200 nm, 80 to 150 nm, 80 to 140 nm, 80 to 130 nm, 80 to 120 nm, or 80 to 110 nm; 100 to 500 nm, 100 to 400 nm, 100 to 350 nm, 100 to 300 nm, 100 to 250 nm, 100 to 220 nm, 100 to 200 nm, 100 to 150 nm, 100 to 140 nm, 100 to 130 nm, 80 to 120 nm, or 80 to 110 nm, but is not limited thereto.

The particles of the present disclosure have a size of nanoscale as described above. Therefore, the particles of the present disclosure are expressed as microparticles or nanoparticles depending on the diameter of the particles.

In a specific embodiment of the present disclosure, the nanoparticles have an average particle size of 144.9±34.5 nm. In another embodiment of the present disclosure, the nanoparticles have an average particle size of 100 nm to 200 nm, 110 nm to 180 nm, 120 nm to 170 nm, 130 nm to 160 nm, or 140 nm to 150 nm.

In one embodiment of the present disclosure, the near-infrared responsive photosensitizer is at least one selected from the group consisting of chlorin e6 (Ce6), porphyrin, photofrin, temoporfin, aminolevulinic acid-induced protoporphyrin IX, motexafin lutetium, padoporfin, padeliporfin, talaporfin (NPe₆), radachlorin, Purlytin, phthalocyanines, Verteporfin, HPPH (photochlor), TPC (5-(4-carboxyphenyl)-10,15,20-triphenyl-2,3-dihydroxychlorin), Chlorin p6 (Cp6), Purpurin-18, purpurinimide, and bacteriochlorin.

In one embodiment of the invention, the covalent bond may be formed between a hydroxyl group of the laminarin and a carboxyl group of the photosensitizer.

In one embodiment of the present disclosure, the hydroxyl group of the laminarin may be a hydroxyl group of the hydroxymethyl group linked to carbon at the position 5 of glucose.

In one embodiment of the present disclosure, the nanoparticles bind to Dectin-1. In another embodiment of the present disclosure, the nanoparticles selectively bind to Dectin-1. In yet another embodiment of the present disclosure, the nanoparticles bind to Dectin-1 on the surface of macrophages.

In a specific embodiment of the present disclosure, the nanoparticles bind to Dectin-1 on the surface of atherosclerotic macrophages. In a more specific embodiment of the present disclosure, the nanoparticles bind to Dectin-1 on the surface of macrophages in atherosclerotic plaques.

As used herein, the term “Dectin-1” means a pattern-recognition receptor that recognizes β-(1,3)-glucan. Dectin-1 is a protein encoded by the human CLEC7A (C-type lectin domain family 7 member A) gene, and is one of group V nonclassical NKCL (NK C-type lectin-like) receptors as a type II-transmembrane protein. It is known that Dectin-1 can specifically recognize at least 9-mer or 10-mer units of β-(1,3)-glucan.

Dectin-1, when bound to a ligand, can mediate various cellular responses such as ligand absorption through endocytosis and phagocytosis, respiratory burst, and production of various cytokines and chemokines. Dectin-1 ligands include β-glucan, laminarin, and the like.

As used herein, the term “β-glucan (β-glucan)” means being insoluble in water among β-D-glucose polysaccharides. β-glucan is naturally present mainly in the cell walls of plants, bacteria and fungi, and is triple-stranded depending on the type. It is also known to lower blood LDL cholesterol and reduce the risk of developing cardiovascular diseases. For example, the β-glucan may include zymosan, curdlan, particulate glucan, etc. having a large or medium molecular weight, but is not necessarily limited thereto.

In the present disclosure, water-insoluble β-glucan and water-soluble laminarin are distinguished.

In another embodiment of the present disclosure, the laminarin specifically binds to Dectin-1.

In one embodiment of the present disclosure, the nanoparticles are absorbed into activated macrophages, foam cells, or atherosclerotic plaques.

The activated macrophages are macrophages that have been activated by exposure to LPS (lipopolysaccharide) and/or LDL (Low-density lipoprotein).

In one embodiment of the present disclosure, the nanoparticles are absorbed into macrophages or foam cells. In another embodiment of the present disclosure, the nanoparticles are absorbed into activated macrophages or foam cells. In another embodiment of the present disclosure, the nanoparticles are absorbed via intracellular inclusion into activated macrophages or foam cells.

In a specific embodiment of the present disclosure, the nanoparticles are more selectively absorbed by activated macrophages or foam cells as compared to macrophages.

In one embodiment of the present disclosure, the nanoparticles are absorbed into the atherosclerotic plaque. In one embodiment of the present disclosure, the nanoparticles are absorbed into the atherosclerotic plaque via intracellular inclusion of macrophages or foam cells activated within the atherosclerotic plaque.

In one embodiment of the present disclosure, the nanoparticles emit a near-infrared fluorescence (NIFR) signal when irradiated with a near-infrared light.

In one embodiment of the present disclosure, the nanoparticles do not emit a near-infrared fluorescence (NIFR) signal before irradiation with a near-infrared light.

In one embodiment of the present disclosure, the nanoparticles generate singlet oxygen (¹0₂) when irradiated with a near-infrared light.

In one embodiment of the present disclosure, the nanoparticles do not generate singlet oxygen (¹0₂) before irradiation with a near-infrared light.

In one embodiment of the present disclosure, the nanoparticles emit a near-infrared fluorescence signal from macrophages, activated macrophages, or foam cells when irradiated with a near-infrared light.

In another embodiment of the present disclosure, the nanoparticles emit a near-infrared fluorescence signal from arteriosclerotic macrophages, arteriosclerotic activated macrophages, or arteriosclerotic foam cells when irradiated with a near-infrared light. In another embodiment of the present disclosure, the nanoparticles emit a near-infrared fluorescence signal from macrophages in the plaque, activated macrophages in the plaque, or foam cells in the plaque when irradiated with a near-infrared light.

In one embodiment of the present disclosure, the nanoparticles generate singlet oxygen (¹0₂) in macrophages, activated macrophages, or foam cells when irradiated with a near-infrared light.

In another embodiment of the present disclosure, the nanoparticles generate singlet oxygen (¹0₂) in arteriosclerotic macrophages, arteriosclerotic activated macrophages, or arteriosclerotic foam cells when irradiated with near-infrared light. In another embodiment of the present disclosure, the nanoparticles generate singlet oxygen (¹0₂) in macrophages in atherosclerotic plaques, activated macrophages in atherosclerotic plaques, or foam cells in atherosclerotic plaques when irradiated with a near-infrared light.

In another embodiment of the present disclosure, the nanoparticles induce apoptosis of macrophages, activated macrophages, or foam cells when irradiated with near-infrared light.

In one embodiment of the present disclosure, the nanoparticles do not induce apoptosis of macrophages, activated macrophages, or foam cells before irradiation with a near-infrared light.

In another embodiment of the present disclosure, the nanoparticles induce apoptosis of arteriosclerotic macrophages, arteriosclerotic activated macrophages, or arteriosclerotic foam cells when irradiated with a near-infrared light. In another embodiment of the present disclosure, the nanoparticles induce apoptosis of macrophages in plaques, activated macrophages in plaques, or foam cells in plaques when irradiated with a near-infrared light.

In one embodiment of the present disclosure, the nanoparticles are systemically circulated within at least 2 hours after intravenous injection. In another embodiment of the present disclosure, the nanoparticles are systemically circulated between 1 hour and 4 hours after intravenous injection.

In one embodiment of the present disclosure, the nanoparticles are present in the body at their highest concentration 5 to 6 hours after intravenous injection. In another embodiment of the present disclosure, the nanoparticles are present in the body at their highest concentration from 3 hours to 12 hours, 4 hours to 10 hours, 4 hours to 8 hours, or 5 hours to 6 hours after intravenous injection.

In one embodiment of the present disclosure, the nanoparticles are present in the body at a concentration of a level that can measure a fluorescence signal up to at least 48 hours after intravenous injection. In one embodiment of the present disclosure, the nanoparticles are present in the body at a concentration of a level that can measure the fluorescence signal for 10 minutes to 48 hours after intravenous injection.

According to another aspect of the present disclosure, there is provided a method for preventing, diagnosing, or treating arteriosclerosis, comprising administering a composition comprising nanoparticles according to an embodiment as an active ingredient to a subject in need thereof.

As used herein, the term “prevention” means prophylactic or protective treatment for a disease or disease condition. As used herein, the term “diagnosis” means confirming the presence or characteristics of a pathological condition. For the purpose of the present disclosure, the diagnosis is to confirm the presence of onset of atherosclerosis, the degree of progression of the disease, or the possibility of developing arteriosclerosis. As used herein, the term “treatment” means a reduction, suppression, amelioration, or eradication of a disease condition.

In one embodiment of the present disclosure, the subject is a mammal or a human.

In one embodiment of the present disclosure, the method for preventing or treating arteriosclerosis further comprises a step of irradiating a near-infrared light after administration of the composition to the subject. In one embodiment of the present disclosure, the method for diagnosing arteriosclerosis comprises a step of irradiating a near-infrared light after administration of the composition to a subject; and a step of measuring the intensity of the near-infrared fluorescence signal emitted from the nanoparticles and comparing it with the result of a normal subject or an arteriosclerosis patient.

In one embodiment of the present disclosure, there is provided a composition for preventing, diagnosing and treating arteriosclerosis comprising nanoparticles according to one embodiment as an active ingredient.

In another embodiment of the present disclosure, there is provided a composition for diagnosing and treating arteriosclerosis comprising the nanoparticles according to one embodiment as an active ingredient.

In one embodiment of the present disclosure, the composition may be a theranostic composition. That is, the present disclosure provides a composition capable of simultaneously or sequentially diagnosing and treating arteriosclerosis comprising nanoparticles according to an embodiment as an active ingredient.

As used herein, the term “theranostic, theragnostic” means next-generation medicine that combines treatment and diagnosis for a specific disease, disease or medical condition. Theranostic is a word derived from a combination of therapeutic and diagnostic. In the present disclosure, the term “theranostic” may be used interchangeably with the term “therapeutic diagnosis”, which is meant to encompass materials, drugs, technologies, devices, etc. that can simultaneously or sequentially treat and diagnose a specific disease, disease, or medical condition.

In one embodiment of the present disclosure, the composition may be a phototheranostic composition. That is, the present disclosure provides a composition capable of simultaneously or sequentially diagnostic and photodynamic treatment of arteriosclerosis comprising nanoparticles according to one embodiment as an active ingredient.

As used herein, the term “phototheranostics” means next-generation medicine that integrates both diagnostic and therapeutic functions for a specific disease, disease or medical condition into a single photosensitizer. In the present disclosure, the term “phototheranostic” can be used interchangeably with the term “photoactivatable theranostic”.

In one embodiment of the present disclosure, the composition further comprises a pharmaceutically acceptable carrier.

The pharmaceutically acceptable carrier of the present disclosure is commonly used in the technical field to which the present disclosure belongs, and is pharmacologically compatible with the nanoparticles, which are the active ingredient of the present disclosure.

Pharmaceutically acceptable carriers of the present disclosure include lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methylcellulose, methylhydroxybenzoate, propylhydroxybenzoate, talc, magnesium stearate, mineral oil, and the like, but is not limited thereto.

The pharmaceutical composition of the present disclosure may further include excipients, stabilizers, diluents, lubricants, wetting agents, sweeteners, flavoring agents, emulsifiers, suspending agents, preservatives, and the like, in addition to the above components. Suitable pharmaceutically acceptable carriers, carriers, excipients, stabilizers or diluents are described in detail in Remington's Pharmaceutical Sciences (19th ed., 1995).

The pharmaceutical composition of the present disclosure may be administered orally or parenterally. For parenteral administration, intravenous injection, subcutaneous injection, intramuscular injection, intraperitoneal injection, percutaneous administration, intracerebral injection, intraspinal injection, and the like can be used.

A suitable dosage of the pharmaceutical composition of the present disclosure varies depending on factors such as formulation methods, administration methods, the patient's age, body weight, sex, severity of diseases, diet, administration time, administration route, excretion rate, and response sensitivity of the patient, and an ordinarily skilled physician can readily determine and prescribe an effective dosage for the desired treatment or prophylaxis. Meanwhile, the dosage of the pharmaceutical composition of the present disclosure is preferably 0.0001-1000 mg/kg (body weight) per day, 0.0001-500 mg/kg (body weight) per day, 0.0001-300 mg/kg per day (body weight), 0.0001-200 mg/kg (body weight) per day, or 0.0001-100 mg/kg (body weight) per day, but is not limited thereto.

The pharmaceutical composition of the present disclosure is formulated using a pharmaceutically acceptable carrier and/or excipient in accordance with the method that can be easily carried out by a person having ordinary knowledge in the technical field to which the invention belongs, so that it can be manufactured in unit dose form or can be manufactured by incorporating into multi-dose containers.

At this time, the formulation may be in the form of a solution, suspension or emulsion in an oil or aqueous medium, or in the form of an X-lot, powder, granules, tablets or capsules, and may further include dispersants or stabilizers.

In one embodiment of the present disclosure, the composition emits a near-infrared fluorescence signal and simultaneously generates a single anti-oxygen when irradiated with a near infrared light.

In one embodiment of the present disclosure, the composition emits a near-infrared fluorescence signal and simultaneously generates a singlet oxygen before irradiation with a near-infrared light.

In one embodiment of the present disclosure, the composition emits a near-infrared fluorescence signal from macrophages, activated macrophages, or foam cells and simultaneously generates a singlet oxygen upon irradiation with a near-infrared light.

In another embodiment of the present disclosure, the composition emits a near-infrared fluorescence signal from arteriosclerotic macrophages, arteriosclerotic activated macrophages, or arteriosclerotic foam cells and simultaneously generates singlet oxygen when irradiated with a near-infrared light. In another embodiment of the present disclosure, the composition emits a near-infrared fluorescence signal from macrophages in atherosclerotic plaques, activated macrophages in atherosclerotic plaques, or foam cells in atherosclerotic plaques and simultaneously generates singlet oxygen upon irradiation with a near-infrared light.

In a specific embodiment of the present disclosure, the composition further emits a near-infrared fluorescence signal from activated macrophages or foam cells and simultaneously further generates singlet oxygen as compared to macrophages when irradiated with a near-infrared light.

In one embodiment of the present disclosure, the composition induces apoptosis of macrophages in atherosclerotic plaques, or reduces the size of atherosclerotic plaques, or reduces macrophage-mediated inflammatory responses, after irradiation with a near-infrared light.

In another embodiment of the present disclosure, the composition induces apoptosis of macrophages in atherosclerotic plaques, activated macrophages in atherosclerotic plaques, or foam cells in atherosclerotic plaques after irradiation with a near-infrared light.

In a specific embodiment of the present disclosure, the composition further induces apoptosis of activated macrophages in atherosclerotic plaques, or foam cells in atherosclerotic plaques as compared to macrophages after irradiation with a near-infrared light.

In one embodiment of the present disclosure, the composition emits a near-infrared fluorescence signal generated from an atherosclerotic plaque, which is distinguished from autofluorescence generated from a normal tissue upon irradiation with near-infrared wavelength light.

As used herein, the term “autofluorescence” means fluorescence naturally emitted by structures such as many proteins which are rich in light-absorbing mitochondria, lysosomes, collagen, elastin, ceroid, lipofuscin, some extracellular lipids, NADPH, flavin, and tryptophan, tyrosine and phenylalinine. Autofluorescence is distinguished from light generated from artificially added fluorescent markers. Most animal tissues exhibit some autofluorescence when excited with ultraviolet light.

Since the composition for preventing, diagnosing, or treating arteriosclerosis contains the above-mentioned nanoparticles as an active ingredient, duplicate contents thereof will be omitted in consideration of the complexity of the present specification.

### Advantageous Effects

The present disclosure provides nanoparticles comprising a near-infrared responsive photosensitizer covalently bonded to laminarin, and a composition for preventing, diagnosing, or treating arteriosclerosis comprising the same as an active ingredient.

The features and advantages of the present disclosure are summarized as follows:

(a) It can improve the atherosclerotic plaque targeting and plaque-penetrating properties relative to conventional photodynamic therapy, and thus can be usefully used for in vivo imaging of atherosclerotic plaque.

(b) Atherosclerotic plaques can be stabilized by inducing macrophage apoptosis in atherosclerotic plaques to reduce the size of atherosclerotic plaques.

(c) The composition comprising the nanoparticles of the present disclosure as an active ingredient can be utilized for prevention, diagnosis and/or treatment of arteriosclerosis in that photodynamic therapy and image diagnosis of arteriosclerotic plaques can be performed simultaneously or sequentially.

### BRIEF DESCRIPTION OF THE DRAWINGS

FIG. 1 is a schematic diagram which shows a method for synthesizing LAM-Ce6 (Laminarin-chlorin e6) using laminarin and chlorin e6 and a crosslinking agent EDC/DMAP, and a state in which LAM-Ce6 forms self-assembled nanoparticles on an aqueous solution.

FIG. 2 is a particle size distribution of LAM-Ce6 according to an embodiment of the present disclosure (a), is a diagram showing a scanning electron microscope image of LAM-Ce6 (scale bar: 500 nm) (b).

FIG. 3 shows the Fourier transform infrared spectral spectrum of laminarin (red) (a), shows the Fourier transform infrared spectral spectrum of LAM-Ce6 (blue) (b), and shows the Fourier transform infrared spectral spectrum of Ce6 (black) (c).

FIGS. 4a, 4b and 4c show the optical characteristics. FIG. 4a shows the ultraviolet and visible spectrum of Ce6 and LAM-Ce6 dissolved in PBS containing 1% Tween 20, and LAM-Ce6 dispersed in PBS. FIG. 4b shows the fluorescence spectra of Ce6 and LAM-Ce6 dissolved in PBS containing 1% Tween 20, and LAM-Ce6 dispersed in PBS. FIG. 4c shows the results of singlet oxygen generation over time after irradiating 670 nm to Ce6 and LAM-Ce6 dissolved in PBS containing 1% Tween 20, and LAM-Ce6 dispersed in PBS.

FIG. 5 shows the results of examining the cytotoxicity through CCK-8 kit at various concentrations of LAM-Ce6 based on Ce6 for macrophages, activated macrophages and foam cells (a) and shows the results of examining the phototoxicity through CCK-8 kit when each cell is treated with various concentrations of Ce6 standards and then irradiated with a 670 nm laser (b).

FIG. 6a shows a fluorescence image comparing the intracellular absorption of LAM-Ce6 to macrophages, activated macrophages, and foam cells.

FIG. 6b shows fluorescence images comparing the degree of intracellular absorption of LAM-Ce6 after first being treated with laminarin serving as an agonist and β-glucan serving as an antagonist in order to compare the ligand-receptor mediated endocytosis by Dectin-1.

FIG. 7 shows in vivo fluorescence images acquired through a small animal imaging device for each time in order to evaluate the distribution and excretion of LAM-Ce6 over time after intravenous injection of LAM-Ce6 into Balb/c nude mice (a) and shows the ex vivo tissue distribution of LAM-Ce6 through fluorescence analysis in liver, lung, spleen, kidney and heart extracted from mice 48 hours after LAM-Ce6 administration (b).

FIG. 8 shows the results of examining the concentrations of alanine aminotransferase (ALP), aspartate aminotransferase (AST), alkaline phosphatase (ALT) and blood uric acid (SUA) in blood by collecting blood 24 hours after intravenous injection of LAM-Ce6.

FIG. 9 shows the results of phototoxicity in skin tissue and H&E staining of dissected skin tissue after 3 days, in a state where the skin was irradiated with a 670 nm laser one hour after intravenous injection of LAM-Ce6 and Ce6 (equiv. of Ce6 5 μM),

FIG. 10 is a schematic diagram of the molecular imaging experimental method protocol for in vivo mouse atherosclerotic plaque target imaging of LAM-Ce6(a) and is a schematic diagram of a multichannel fluorescence microscope in vivo molecular imaging system (Customized multichannel IVFM imaging system) (b).

FIG. 11 shows the results of fluorescence images acquired by an in vivo molecular imaging system of the carotid artery region under a multi-channel fluorescence microscope 48 hours after intravenous injection of LAM-Ce6 4 mg/kg (equiv. of Ce6) (a) and is a diagram showing the result of a fluorescent image of a tissue cross-section in which the extracted carotid artery was sectioned to 10 μm (b).

FIG. 12 is a schematic diagram of a protocol for viewing the therapeutic effect of LAM-Ce6 nanoparticles by photodynamic therapy of mouseatherosclerotic plaques.

FIG. 13 is the fluorescence image results of atherosclerotic plaques acquired with a multi-channel fluorescence microscope in vivo molecular imaging system one week after irradiating a laser (670 nm, 1 W/cm², 100 J/cm²) to the carotid artery atherosclerotic plaque 48 hours after intravenous injection of LAM-Ce6 4 mg/kg (equiv. of Ce6) (a), is a diagram showing a fluorescence image of a tissue cross-section in which the extracted carotid artery was sectioned to 10 μm and an image stained with F4/80, Dectin-1, H&E, and ORO (b).

FIG. 14 is a schematic diagram of an FRI imaging method protocol for in vivo rabbit atherosclerotic plaque target imaging of LAM-Ce6 nanoparticles.

FIG. 15 is a schematic diagram of an OCT-NIRF system and an intravascular imaging system for in vivo imaging using LAM-Ce6 nanoparticles in a rabbit arteriosclerosis model.

FIG. 16 shows the image results acquired from the aortic atherosclerotic plaque 48 hours after the injection of 4 mg/kg (equiv. of Ce6) of LAM-Ce6 nanoparticles into the ear vein in a rabbit arteriosclerosis model (a) and is a diagram showing the results of a fluorescence image of a tissue cross-section in which the extracted carotid artery was sectioned to 10 μm (b).

FIG. 17 is a schematic diagram of a protocol for observing the therapeutic effect of LAM-Ce6 nanoparticles by photodynamic therapy of rabbit arteriosclerotic plaque.

FIG. 18 is fluorescence images obtained from aortic atherosclerotic plaques 48 hours after injection of LAM-Ce6 nanoparticles 4 mg/kg (equiv. of Ce6) into the ear vein (uptake) and after photodynamic therapy (after PDT) in a rabbit arteriosclerosis model (a), is a fluorescence image obtained in the forward and flip directions in the arteriosclerotic plaque after photodynamic therapy (b) and is a view showing a fluorescence image and a TUNEL staining image of a tissue cross-section in which the carotid artery extracted according to laser irradiation was sectioned to 10 μm (c).

FIG. 19 shows the results of histological analysis of the degree of atherosclerotic plaque retraction and stabilization through phototherapy. (a) shows that the inflammatory signal was greatly reduced after 4 weeks of phototherapy progress. (b) shows the results of measurement of RAM11 expression level showing the degree of reduction in the size of macrophages and arteriosclerotic plaques, and PSR staining images showing the amount of collagen.

### DETAILED DESCRIPTION

Hereinafter, the present disclosure will be described in more detail with reference to examples. It would be obvious to those skilled in the art that these examples are intended to be more concretely illustrative and the scope of the present disclosure as set forth in the appended claims is not limited to or by the examples.

### Preparation Example: Synthesis of Dectin-1 Target Nanoparticles (LAM-Ce6 Nanoparticles)

LAM-Ce6 nanoparticles for targeting Dectin-1 expressed in atherosclerotic plaques were synthesized as shown in the following Reaction Scheme.

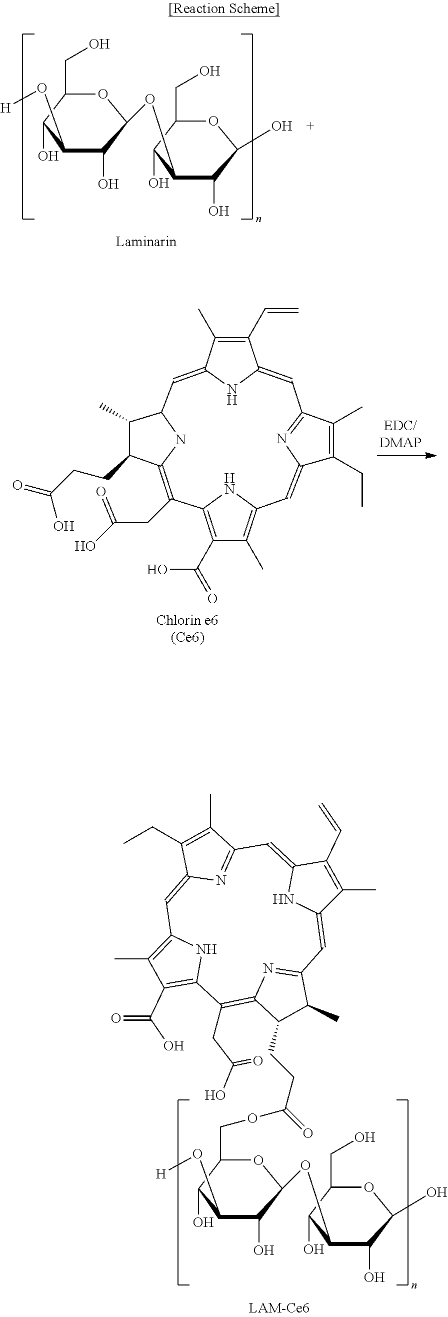

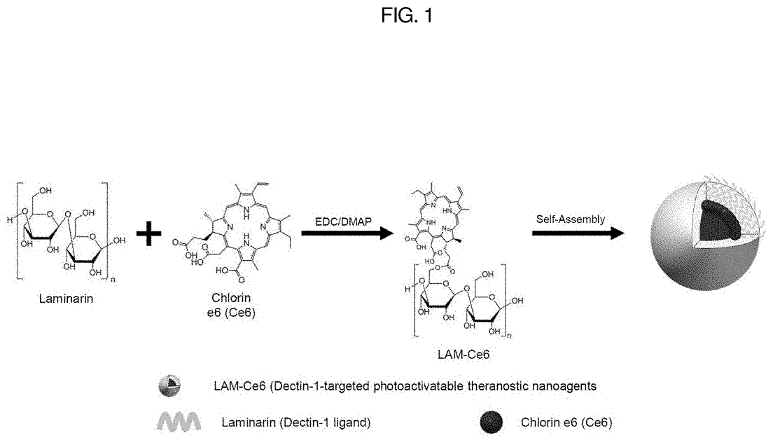

Laminarin (Sigma-Aldrich) (0.1 g) was completely dissolved in 20 mL of DMSO at 60° C. for 24 hours. EDC (1-(3-Dimethylaminopropyl)-3-ethylcarbodiimide hydrochloride, Sigma-Aldrich) (0.0213 g), DMAP (4-Dimethylaminopyridine, Sigma-Aldrich) (0.0136 g) and Ce6 (Chlorin e6, Frontier Scientific) (0.0333 g) were dissolved in the dissolved laminarin solution, and the reaction was carried out. EDC and DMAP were further added twice, and the reaction was carried out for 2 days. The solution obtained after the reaction was dialyzed in 50% DMSO aqueous solution (dimethyl sulfoxide, Samjeon Chemical and Sigma-Aldrich) for 5 days using a regenerated cellulose dialysis membrane (1 kD Molecular weight cut-off (MWCO), Spectrum Laboratories) for 5 days, and further dialyzed for 2 days, purified and then lyophilized (see FIG. 1). The result was referred to as LAM-Ce6 (Laminarin-Ce6). LAM represents the laminarin moiety and Ce6 represents the chlorine e6 moiety.

### Example 1: Characterization of LAM-Ce6 nanoparticles

1.1: Confirmation of Size, Polydispersity Index, Z-Average Size and Morphology of LAM-Ce6 Nanoparticles

1.1.1: Nanoparticle Size, Polydispersity Index and Z-Average Size

The dried LAM-Ce6 was dispersed in distilled water at 0.1 mg/mL, and then analyzed with a particle size analyzer (dynamic light scattering, DLS).

1.1.2: Nanoparticle Morphology

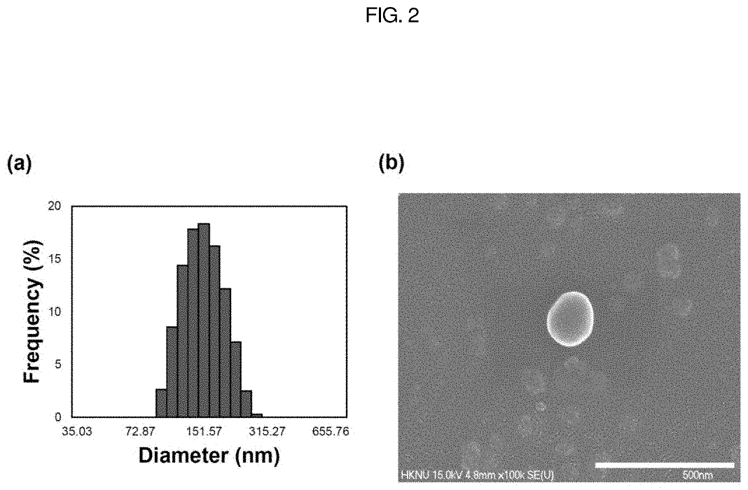

1 mg of LAM-Ce6 was dispersed in distilled water, and the dispersed solution was dropped on a glass cover slip, allowed the sample to air dry and then coated with platinum. The analysis was performed using a field emission scanning electron microscope (FE-SEM) operating at 15.0 kV. The results are shown in FIG. 2(a) and FIG. 2(b).

1.1.3: Results

As shown in FIG. 2, the Z-average size of LAM-Ce6 dispersed in distilled water is 137 nm, the average size of the particles is 144.9±34.5 nm, and the polydispersity index (PDI) is 0.135 (see FIG. 2(b)). In addition, it was confirmed that LAM-Ce6 has a spherical shape (see FIG. 2(b)). This is a result proving that when LAM-Ce6 exists in an aqueous solution, hydrophilic LAM is rearranged outside the particle and hydrophobic Ce6 is re-arranged inside the particle to form a self-assembled nanostructure.

1.2: Confirmation of Binding of LAM and Ce6 of LAM-Ce6 Nanoparticles

Laminarin, resulting LAM-Ce6 and Ce6, was analyzed by Fourier transform infrared spectroscopy (FT-IR). For FT-IR analysis, samples were prepared in the form of potassium bromide (KBr) pellets. FT-IR spectra were acquired with a resolution of 4 cm⁻¹ in the range of 4,000 cm⁻¹ to 400 cm⁻¹.

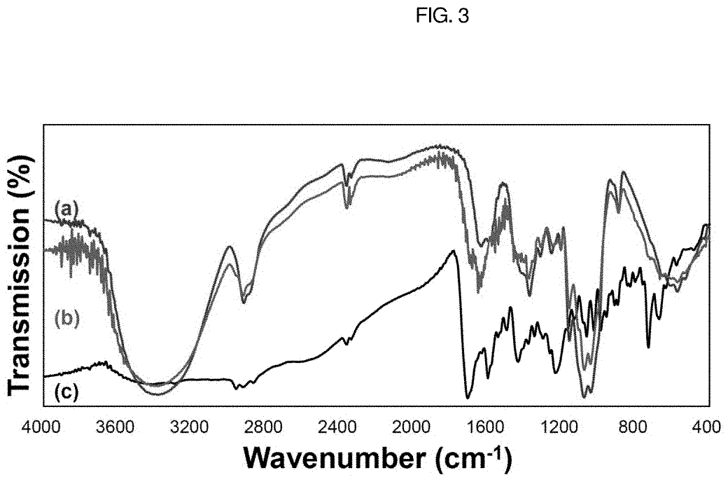

The results are shown as laminarin (red) in FIG. 3(a), LAM-Ce6 (blue) in FIG. 3 (b), and Ce6 (black) in FIG. 3 (c), respectively.

The peak of the N—H bond appearing at 2961 cm⁻¹ and the peak of the C═H bond appearing at 1709 cm⁻¹ of the Ce6 spectrum shown as Ce6 (black) in FIG. 3(c) appear at 2947 cm⁻¹ and 1684 cm⁻¹ of the LAM-Ce6 spectrum shown as LAM-Ce6 (blue) in FIG. 3(b). The result proves that LAM and Ce6 are properly bound.

1.3: Confirmation of Structure Formation of LAM-Ce6 Nanoparticles

UV/Vis (ultraviolet and visible light) and fluorescence spectra were analyzed to determine whether LAM-Ce6 forms nanoparticles. For analysis, LAM-Ce6 was dissolved in PBS so as to be 5 μM based on Ce6, and for comparison, LAM-Ce6 and Ce6 were prepared in PBS containing 1% Tween 20 at the same concentration. UVNis absorbance was acquired from 300 nm to 800 nm, and the fluorescence intensity was acquired from 600 nm to 800 nm.

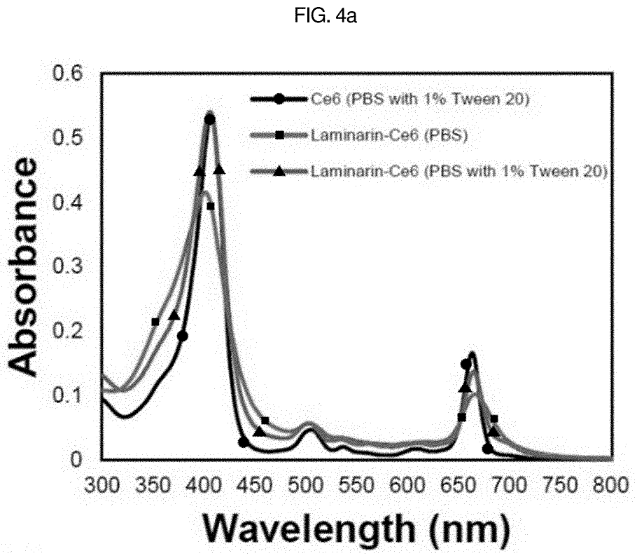

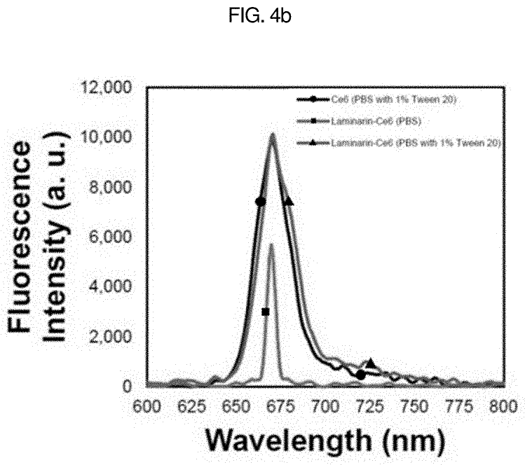

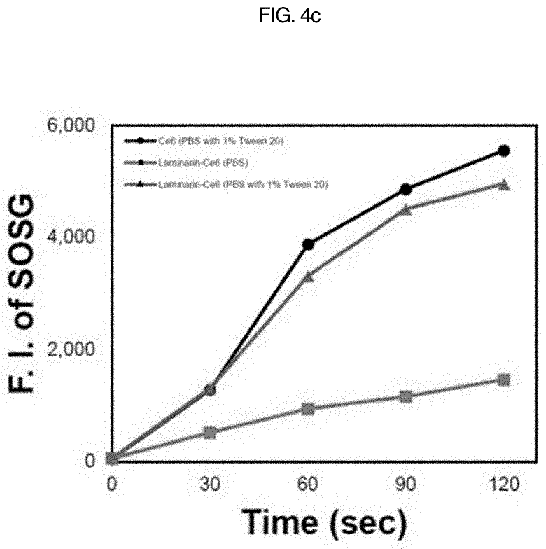

The results are shown in FIG. 4a and FIG. 4b.

As shown in FIG. 4a and FIG. 4b, the absorbance and fluorescence intensity of Ce6 (PBS with 1% Tween 20) and LAM-Ce6 (PBS with 1% Tween 20) were similar to each other, whereas both the absorbance and fluorescence intensity of LAM-Ce6 (PBS) showed low results. The LAM-Ce6 (PBS) of the present disclosure exhibited reduced UVNis wavelength absorbance and fluorescence intensity, as compared to Ce6 (PBS with 1% Tween 20) and LAM-Ce6 (PBS with 1% Tween 20), in which the formation of self-assembled nanoparticles was inhibited by a surfactant, whereby it can be confirmed that the LAM-Ce6 (PBS) of the present disclosure forms the nanoparticle that the Ce6 is located inward. That is, from the above results, it was verified that Ce6 forms a LAM-Ce6 nanostructure that the Ce6 is located inward in an aqueous solution.

1.4: Confirmation of Singlet Oxygen Generation of LAM-Ce6 Nanoparticles

The singlet oxygen sensor green (SOSG, Invitrogen) fluorescence intensity was analyzed to determine whether singlet oxygen was generated when irradiating LAM-Ce6 with a laser having a near-infrared light (670 nm wavelength). For analysis, PBS at pH 7.4 was saturated with oxygen for 30 min and LAM-Ce6 was dissolved at 5 μM based on Ce6. For comparison, Ce6 and LAM-Ce6 were dissolved in oxygen-saturated PBS containing 1% Tween 20 at the same concentration. All three solutions contained 1 μM SOSG. The fluorescence intensity of SOSG (Ex(excitation)/Em(emission): 504 nm/525 nm) was measured by irradiating a laser (670 nm, 50 mW/cm²) every 30 seconds from 0 to 120 seconds.

The results are shown in FIG. 4c.

As shown in FIG. 4c, the SOSG fluorescence intensity of Ce6 (PBS with 1% Tween 20) and LAM-Ce6 (PBS with 1% Tween 20) showed the tendency to increase similarly to each other, whereas LAM-Ce6 (PBS) showed a relatively low fluorescence intensity. That is, the LAM-Ce6 (PBS) of the present disclosure generates singlet oxygen when irradiated with a near-infrared light, but it generates significantly lower singlet oxygen as compared to Ce6 (PBS with 1% Tween 20) and LAM-Ce6 (PBS with 1% Tween 20), in which the formation of self-assembled nanoparticles was inhibited by the surfactant. Therefore, the above results suggest that the exposure of singlet oxygen to the non-target is low when irradiated with a near-infrared light after injection into the body.

The present inventors were able to prove from the above results that LAM-Ce6 forms nanoparticles, and demonstrated that when irradiated with a near-infrared light to LAM-Ce6, singlet oxygen was generated and the effect of phototoxicity was small during circulation in the body.

### Example 2. Evaluation of Cytotoxicity and Phototoxicity of LAM-Ce6 Nanoparticles

2.1: Evaluation of Cytotoxicity of LAM-Ce6 Nanoparticles

The in vitro cytotoxicity of LAM-Ce6 against macrophages, activated macrophages and foam cells was evaluated using a CCK-8 assay kit (Cell Counting Kit-8, Dojindo Molecular Technologies, Inc.).

Specifically, RAW 264.7 cells (monocytes and macrophages of mouse BALB/c, Korea Cell Line Bank, KCLB, No. 40071) were cultured in RPMI 1640 (Roswell Park Memorial Institute 1640 media, Welgene Inc.) containing 10% FBS and 1% penicillin-streptomycin at 37° C. Subcultured RAW 264.7 cells (2×10⁴ cells/well) were transferred to 96-well culture plates and allowed to adhere for 1 day. To prepare activated macrophages and foam cells, lipopolysaccharide (LPS, Sigma-Aldrich) (200 ng/mL) and low-density lipoprotein (LDL, Sigma-Aldrich) (100 μg/mL) were treated for 1 day. Macrophages, activated macrophages and foam cells were treated with various concentrations of LAM-Ce6 (1 μM, 2 μM, 5 μM, 10 μM and 20 μM) for 4 hours. After 2 hours of material treatment, CCK-8 solution was added to the cells, and the cells were further cultured for 2 hours, and the absorbance was measured at 450 nm.

2.2: Evaluation of Phototoxicity of LAM-Ce6 Nanoparticles

A near-infrared laser (670 nm, 50 mW/cm²) was irradiated after cell culture and material treatment in the same manner as in Example 2.1. After 16 hours, CCK-8 solution was added to the cells, and the cells were further cultured for 2 hours, and then the absorbance was measured at 450 nm.

2.3: Results

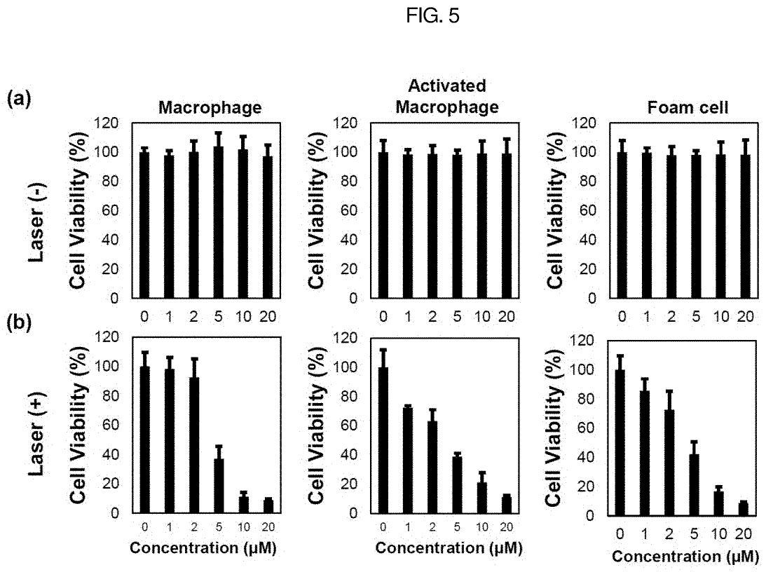

FIG. 5(a) shows the cytotoxicity evaluation results, and FIG. 5(b) shows the phototoxicity evaluation results.

When the near-infrared laser was not irradiated as shown in FIG. 5(a), the cell viability (%) according to the LAM-Ce6 concentration was not significantly different from the case where LAM-Ce6 was not added in all cells. This means that LAM-Ce6 itself has no toxicity against macrophages, activated macrophages and foam cells.

However, when irradiated with a near-infrared laser as shown in FIG. 5 (b), the viability of macrophages, activated macrophages and foam cells was decreased in a concentration-dependent manner. From the above results, the inventors confirmed that LAM-Ce6 is phototoxic to these cells.

### Example 3. Confirmation of Intracellular Absorption of LAM-Ce6 Nanoparticles

It is necessary to confirm whether laminarin modified by binding to Ce6 also absorbs LAM-Ce6 nanoparticles into cells through Dectin-1 and ligand-receptor binding.

Concentration-Dependent Intracellular Absorption of LAM-Ce6

To evaluate the intracellular absorption of LAM-Ce6, RAW 264.7 cells (1×10⁵ cells/well) were cultured in 4-well chamber slides. Next, activated macrophages and foam cells were established by treatment with LPS and LDL. When macrophages were treated with LPS and LDL, macrophages were activated, and the activated macrophages were differentiated to become foam cells. Macrophages, activated macrophages and foam cells were treated with LAM-Ce6 (1 μM, 5 μM and 10 μM) and incubated for 4 hours. Then, the cells were washed 3 times with DPBS and was fixed with 3.7% formalin for 30 minutes. The fixed cells were treated with DAPI to stain the cell nuclei. Then, the cells were imaged using a fluorescence microscope.

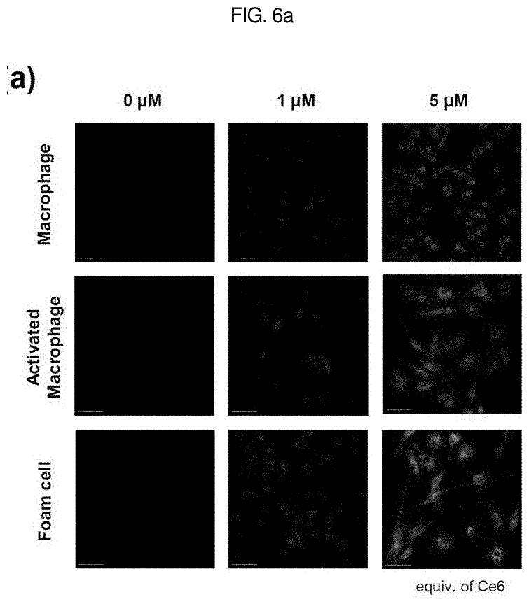

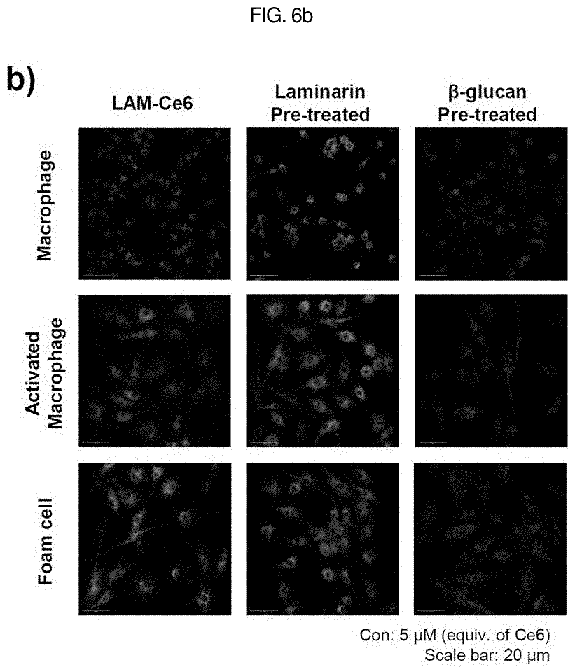

The results are shown in FIG. 6a.

As shown in FIG. 6a, the concentration-dependent NIRF (near-infrared fluorescence) signal of LAM-Ce6 was increased in activated macrophages and foam cells as compared to macrophages. That is, NIRF (near-infrared fluorescence) signal increased in a concentration-dependent manner of LAM-Ce6 in activated macrophages and foam cells as compared to macrophages. That is, the LAM-Ce6 nanoparticles according to the invention were selectively absorbed by activated macrophages and foam cells as compared to non-activated macrophages.

Ligand-Receptor Mediated Intracellular Absorption of LAM-Ce6

To evaluate the ligand-receptor mediated intracellular absorption of LAM-Ce6, RAW 264.7 cells (1×10⁵ cells/well) were cultured in 4-well chamber slides. Next, activated macrophages and foam cells were established by treatment with LPS and LDL. Laminarin (1 mg/mL) acting as an agonist and β-glucan acting as an antagonist (1 mg/m L, β-1,3-Glucan from Euglena gracilis, Sigma Aldrich, CAS Number: 9051-97-2) against macrophages, activated macrophages and foam cells were treated for 1 hour each, then treated with LAM-Ce6 (1 μM, 5 μM, and 10 μM) and incubated for 4 hours. Cells were washed with DPBS, fixed with 3.7% formalin, and then the cell nuclei were stained with DAPI (4′,6-diamidino-2-phenylindole, Dana Korea and GBI Labs).

The results are shown in FIG. 6b.

As shown in FIG. 6b, the fluorescence signal of LAM-Ce6 increased when pre-treated with laminarin, whereas the fluorescence signal of LAM-Ce6 decreased when pre-treated with β-glucan.

These results were resulted from the activation of ligand-receptor mediated endocytosis by Dectin-1, resulting in increased intracellular absorption of LAM-Ce6 when pre-treated with laminarin, and resulted from the inhibition of ligand-receptor-mediated endocytosis by Dectin-1, resulting in decreased intracellular absorption of LAM-Ce6 when pre-treated with 1-glucan.

From the above results, the present inventors demonstrated that LAM-Ce6 was absorbed into cells by Dectin-1.

### Example 4. Evaluation of Biodistribution and Excretion of LAM-Ce6 Nanoparticles

To evaluate the distribution and excretion of LAM-Ce6 in the body over time, Balb/c mice were injected with LAM-Ce6 (2 mg/kg/100 μL) at 2 mg/kg of mouse body weight via tail vein. In vivo near-infrared fluorescence images were acquired using an imaging device for small animals at 10 minutes, 30 minutes, 1 hour, 2 hours, 3 hours, 4 hours, 5 hours, 6 hours, 12 hours, 24 hours and 48 hours after injection.

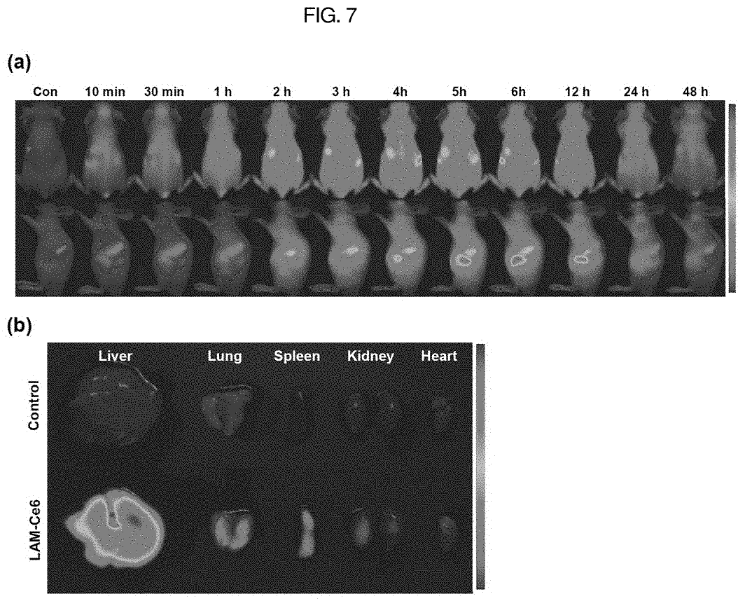

The results are shown in FIG. 7(a).

As shown in FIG. 7(a), a strong NIRF signal was observed up to 6 hours after LAM-Ce6 injection, after which the signal intensity gradually decreased. However, the NIRF signal remained visible for up to 2 days, which is a result demonstrating that the circulation period in the body of nanoparticles was extended.

In addition, 48 hours after LAM-Ce6 administration, liver, lung, spleen, kidney and heart, known as organs in which a large number of macrophages exist, were extracted from Balb/c mice, and ex vivo fluorescence images were acquired and analyzed.

The results are shown in FIG. 7(b).

As shown in FIG. 7(b), 48 hours after LAM-Ce6 injection, the NIRF signal was still detected in liver tissue due to the amphiphilic nature of LAM-Ce6 nanoparticles.

From the above results, it can be confirmed that when upon injection of the LAM-Ce6 nanoparticles of the present disclosure, nanoparticles circulate throughout the body for up to 6 hours and start to discharge after 6 hours, exist in the body of the mouse for at least 2 days, are distributed in the lungs, spleen and kidney tissues in the body, and particularly, are mainly distributed in liver tissue.

### Example 5. Confirmation of the Presence of Liver Function Damage by LAM-Ce6 Nanoparticles

From Example 4, it was confirmed that LAM-Ce6 nanoparticles were mainly distributed in liver tissue when injected into the body. Thus, whether LAM-Ce6 nanoparticles cause damage to liver function was confirmed by blood concentration analysis of Alanine aminotransferase (ALT), aspartate amiotransaminase (AST), Alkaline aminophosphatase (ALP) and Serum uric acid (SUA) 24 hours after LAM-Ce6 injection.

To analyze the concentrations of ALP, AST, ALT and SUA in blood after LAM-Ce6 injection, 2 mg LAM-Ce6 per body weight(kg) (2 mg/kg/100 μL), 4 mg LAM-Ce6 per body weight(kg) (4 mg/kg/100 μL) and 6 mg of LAM-Ce6 per body weight(kg) (6 mg/kg/100 μL) (equiv. of Ce6) were injected into 8-week-old Balb/c mice by tail vein injection. 24 hours after the drug injection, blood was collected through the vena cava. The serum was obtained by centrifugation (1500×g, 10 min, 4° C.). The concentrations of ALP, AST, ALT and SUA in serum were measured through blood chemistry assay.

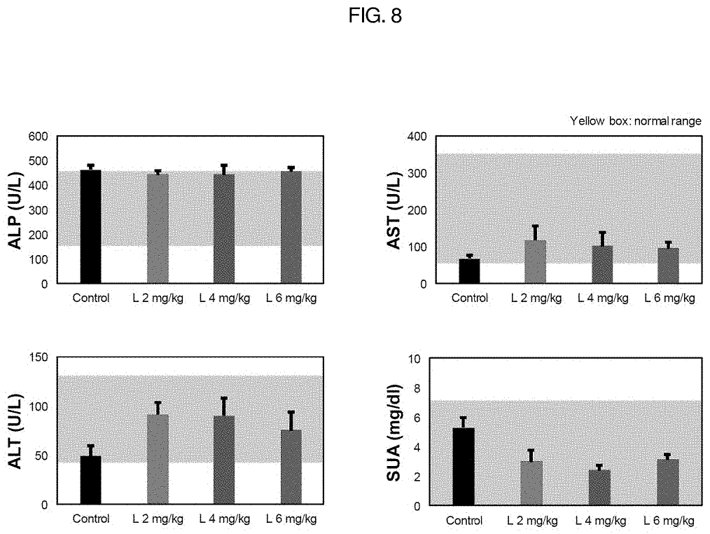

The results are shown in FIG. 8.

As shown in FIG. 8, at all LAM-Ce6 injection concentrations, ALP, AST, ALT, and SUA were all within the normal range (yellow box) of concentrations. This is the result of confirming that the LAM-Ce6 nanoparticles of the present disclosure, which are mainly distributed in liver tissue during intravenous injection, do not cause significant damage to liver function.

### Example 6. Evaluation of Skin Phototoxicity of LAM-Ce6 Nanoparticles

To evaluate the skin phototoxicity of LAM-Ce6, 8-week-old Balb/c mice were intravenously injected with 2 mg of LAM-Ce6 (2 mg/kg/100 μL) per body weight (kg) (equiv. of Ce6) and the same concentration of free Ce6 (prepared by dissolving Ce6 in a small amount of DMSO and adding PBS) as a control group. One hour after injection, a near-infrared (670 nm) laser (50 mW/cm²) was irradiated for 5 minutes. Where erythema occurred on the 2^{nd} and 3^{rd} days was confirmed and the skin tissue was peeled off and H&E (hematoxylin and eosin) staining was performed.

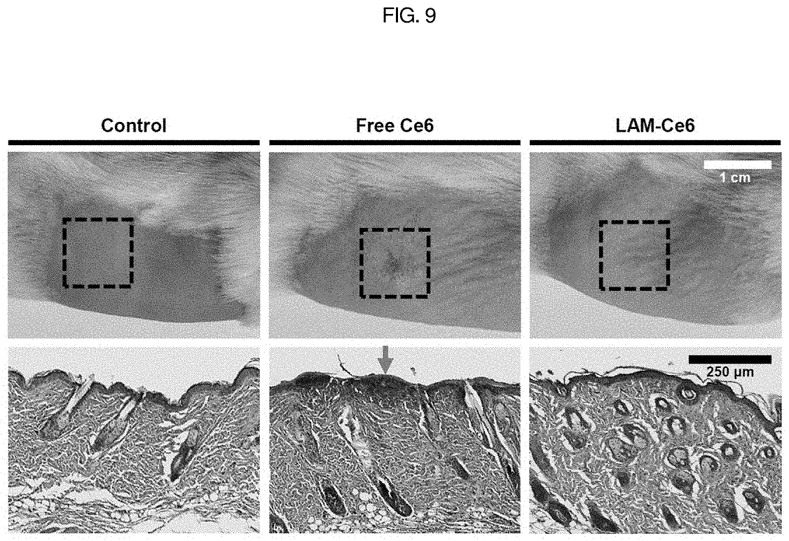

The results are shown in FIG. 9.

As shown in FIG. 9, since the erythema occurred in the mouse injected with free Ce6 (free Ce6), unlike injected with LAM-Ce6 (LAM-Ce6), it can be confirmed that the LAM-Ce6 did not cause skin phototoxicity because it exited in the form of nanoparticles, unlike free Ce6.

### Example 7. LAM-Ce6 Nanoparticles Target Macrophages in Atherosclerotic Plaques in Mice In Vivo

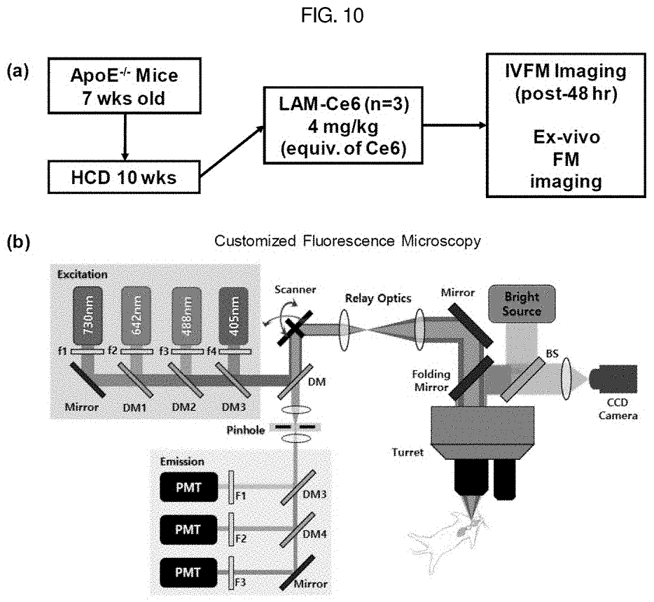

To evaluate the properties of LAM-Ce6 against macrophage targets in atherosclerotic plaques, an in vivo molecular imaging system was used in a mouse model in which arteriosclerosis was induced. FIG. 10 (a) shows the experimental method protocol, and FIG. 10 (b) shows a schematic diagram of a customized multi-channel in vivo molecular imaging system.

Atherosclerosis Mouse Model Formation and LAM-Ce6 Nanoparticle Injection Method

Similar to the protocol shown in FIG. 10(a), a high-cholesterol diet (HCD) was supplied to 7-week-old ApoE^{./.} genetically modified mice (C.KOR/StmSlc-Apoe^{shl}, imported from Japan SLC, purchased from Central Laboratory Animals Co., Ltd.) for a total of 10 weeks to produce atherosclerotic plaques. 48 hours after injection of LAM-Ce6 (4 mg/kg) (equiv. of Ce6) into the tail vein, signals in the bilateral carotid arteriosclerotic plaques of mice were measured through multichannel fluorescence microscopy in vivo molecular imaging (IVFM) in the atherosclerotic plaques. After obtaining the IVFM image, the carotid artery was extracted and sectioned into a size of 10 μm, and then a fluorescence image of the tissue section (ex vivo FM imaging) was obtained.

Multi-Channel Fluorescence Microscopy In Vivo and Ex Vivo Fluorescence Imaging Methods

In addition, as shown in the schematic diagram of FIG. 10(b), molecular imaging was performed through a customized multichannel IVFM imaging system. Specifically, in the imaging system, several laser wavelengths are generated by an excitation module and irradiated to atherosclerotic plaques through an optical system to activate LAM-Ce6. The laser emitted from LAM-Ce6 in the atherosclerotic plaque was delivered to the emission module through the same optical system, and the individual laser lights were separated and measured.

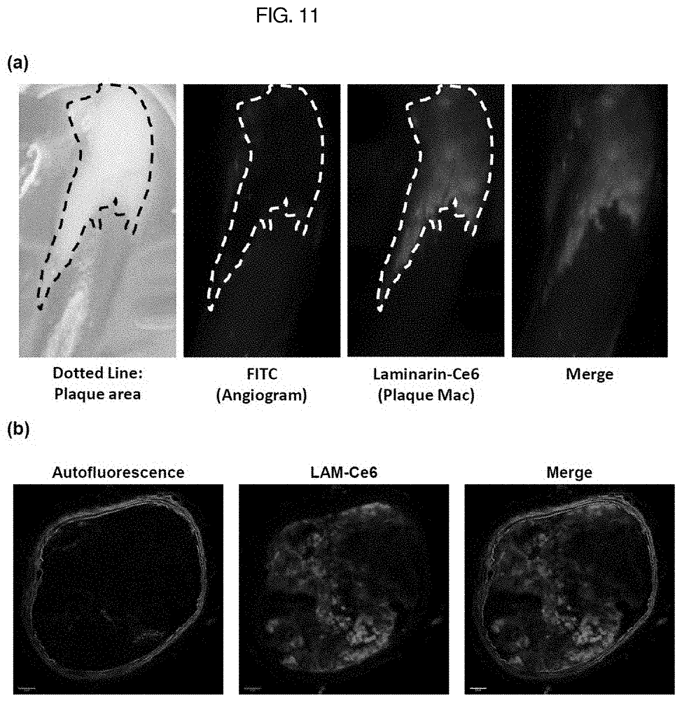

The IVFM image is shown in FIG. 11(a), and the fluorescence image of the tissue section is shown in FIG. 11(b).

Confirmation of Absorption Rate of LAM-Ce6 Nanoparticles in Arteriosclerotic Plaque

As shown in FIG. 11(a), 48 hours after LAM-Ce6 injection, the Ce6 near-infrared signal (red) was measured to be very high in the atherosclerotic plaque (region within the dotted line). Accordingly, it was confirmed that the absorption rate of LAM-Ce6 in the atherosclerotic plaque was high. The FITC (Fluorescein isothiocyanate) shows an angiogram of the carotid artery where autofluorescence occurred at the wavelength corresponding to FITC, the Plaque Mac represents macrophages of sclerotic plaques, and the Merge shows the overlay of fluorescence images of FITC and Laminarin-Ce6.

As shown in FIG. 11(b), in the image obtained from the cross-section of the atherosclerotic plaque in which the extracted carotid artery was sectioned to 10 μm, it was confirmed that the near-infrared signal (red) of LAM-Ce6 penetrated up to a deep place into the plaque. The autofluorescence represents the intrinsic fluorescence signal of carotid elastic fibers emitted by the FITC signal, and the LAM-Ce6 shows a fluorescence signal by near-infrared rays. The Merge represents the overlay of two fluorescence signals.

### Example 8. In Vivo Mouse Atherosclerotic Plaque Photodynamic Therapeutic Effect of LAM-Ce6 Nanoparticles

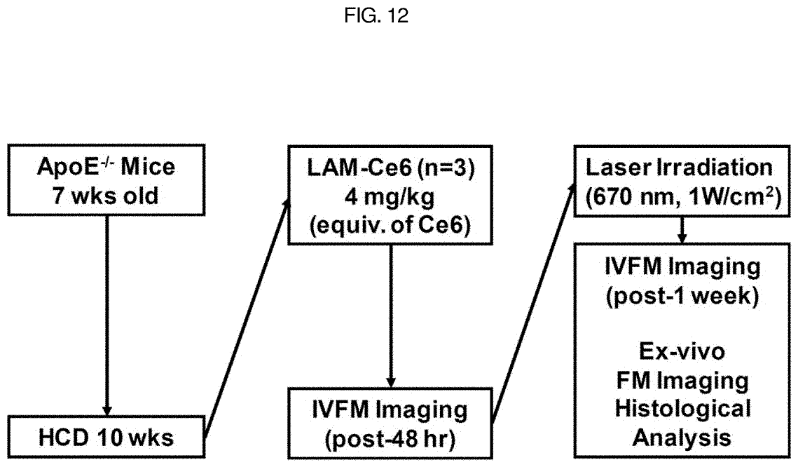

A protocol for confirming the effect of photodynamic therapy (PDT) on atherosclerotic plaques is shown in FIG. 12.

In Vivo Image Analysis of Photodynamic Therapeutic Effects

To evaluate the photodynamic therapeutic effect of LAM-Ce6 in atherosclerotic plaques, atherosclerotic plaques were generated in 7-week-old ApoE^{./.} transgenic mice through a high-cholesterol diet (HCD) for a total of 10 weeks. Then, the in vivo imaging was performed 48 hours after LAM-Ce6 (4 mg/kg) (equiv. of Ce6) was injected into the tail vein. A laser (670 nm, 1 W/cm 2, 100 J/cm²) was irradiated to the atherosclerotic plaque. On day 5, in vivo imaging was performed 48 hours after LAM-Ce6 (4 mg/kg) (equiv. of Ce6) was injected into the tail vein (1 week after laser irradiation).

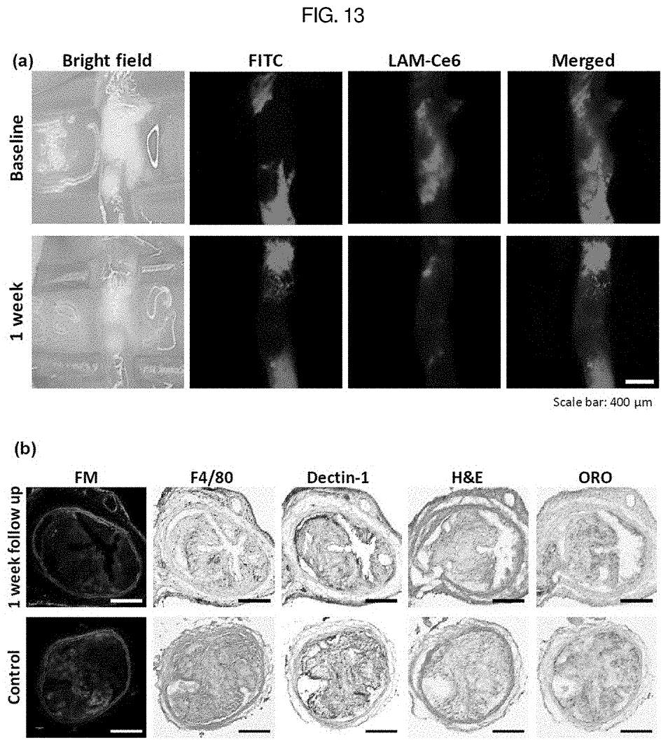

The results are shown in FIG. 13 (a).

As shown in FIG. 13 (a), the size of atherosclerotic plaques decreased one week (1 week) after laser irradiation as compared to before (baseline) laser irradiation (see Bright field), and the NI RF signal also decreased (see LAM-Ce6).

From the above results, the present inventors found that the photoactivation of LAM-Ce6 can restore atherosclerotic plaque, and the inflammation of atherosclerotic plaques can be reduced one week after laser irradiation.

Histological Analysis of Phototherapy Effect

To histologically analyze the effect of phototherapy, the extracted carotid artery was sectioned to 10 μm to confirm the fluorescence image, and F4/80, Dectin-1, H&E and ORO staining was performed. The F4/80 is a membrane protein known as a marker of mature mouse macrophages, and antibodies to F4/80 were obtained from Santa Cruz Biotechnology (SC-52664, Santa Cruz Biotechnology). The ORO (Oil Red 0) is a lysochrome diazo dye used for staining triglycerides and lipids, and was obtained from ScyTek (Oil Red 0 Stain Kit (For Fat), ORK-2, LOT #: 57902, ScyTek).

The results are shown in FIG. 13(b).

As shown in FIG. 13(b), the fluorescence signal decreased in the subjects treated with LAM-Ce6 light as compared to the control group, which was a group injected only with LAM-Ce6 nanoparticles without irradiating a laser (see FM), and Dectin-1 and ORO stained areas were also reduced as compared with the control group.

Overall, from the results, the inventors confirmed that LAM-Ce6 photoactivation can effectively attenuate macrophage-mediated inflammatory response and convert plaques to a good stable state after 1 week of phototherapy.

### Example 9. LAM-Ce6 Nanoparticles Target Macrophages in Rabbit Atherosclerotic Plaque In Vivo

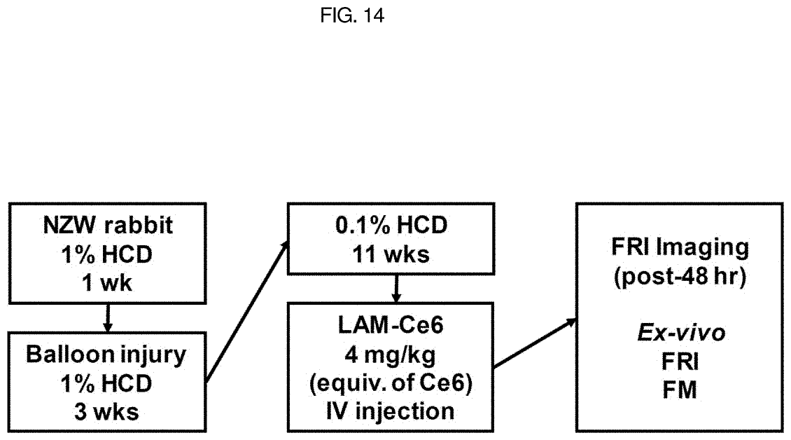

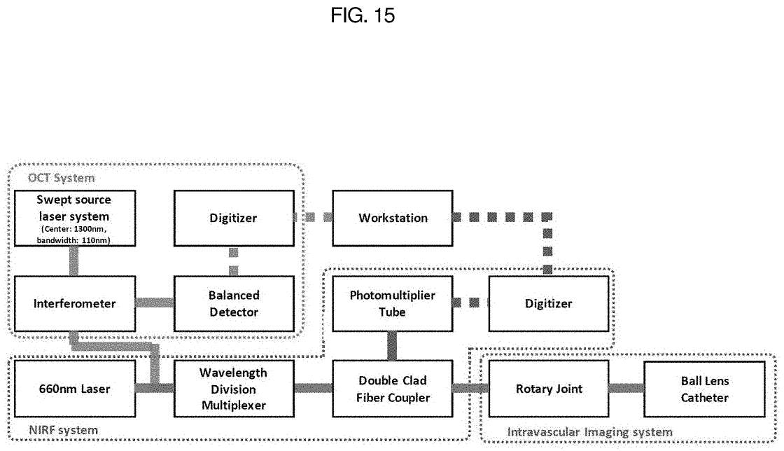

To evaluate the properties of LAM-Ce6 nanoparticles against macrophage targets in atherosclerotic plaques, the OCT-NIRF was used in a rabbit model in which atherosclerosis was induced. FIG. 14 shows the experimental method protocol, and FIG. 15 shows a schematic diagram of the OCT-NIRF system.

Atherosclerosis Rabbit Model Formation and LAM-Ce6 Nanoparticle Injection Method

Similar to the protocol shown in FIG. 14, a 1% cholesterol diet (HCD) was fed to a NZW rabbit for 1 week, and then the aorta was subjected to balloon injury. After maintaining a 1% cholesterol diet for 3 weeks, 0.1% cholesterol diet was fed for 11 weeks to induce atherosclerotic plaque. The aorta was extracted 48 hours after injecting 4 mg/kg of LAM-Ce6 nanoparticles (equiv. of Ce6) into the ear vein. Signals in rabbit aortic plaques were measured by FRI. Further, after sectioning the arteriosclerotic plaque with 10 μm from the extracted carotid artery, fluorescence images of tissue sections (ex vivo FM imaging) were obtained.

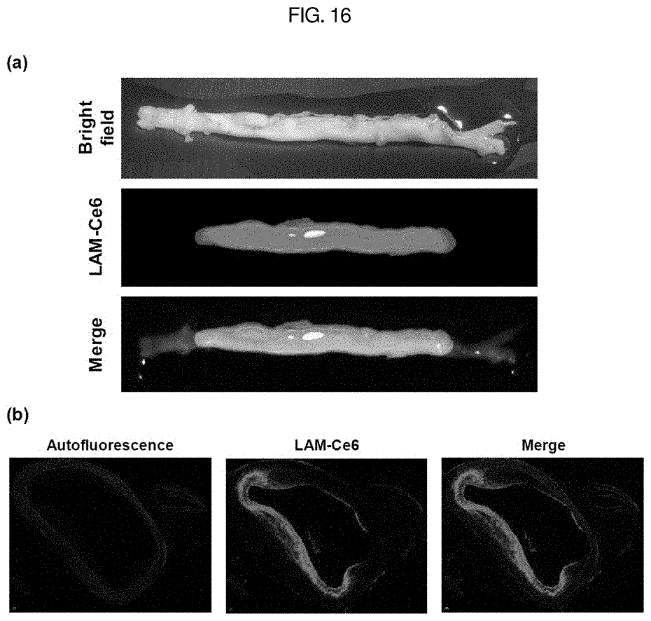

The results are shown in FIG. 16.

Confirmation of Absorption Rate of LAM-Ce6 Nanoparticles in Arteriosclerotic Plaque

As shown in FIG. 16(a) and FIG. 16(b), the Ce6 near-infrared signal (red) was measured to be very high in atherosclerotic plaques 48 hours after LAM-Ce6 nanoparticle injection. Thereby, it was confirmed that the absorption rate of LAM-Ce6 nanoparticles in the atherosclerotic plaque was high.

As shown in FIG. 16(b), it can be confirmed that in the image obtained from the cross-section of the atherosclerotic plaque in which the extracted carotid artery was sectioned at 10 μm, the near-infrared signal (red) of LAM-Ce6 nanoparticles penetrated up to a deep place into the arteriosclerotic plaque. The autofluorescence refers to the intrinsic fluorescence signal of carotid elastic fibers emitted by the FITC signal, the LAM-Ce6 shows a fluorescence signal by near-infrared rays, and the Merge represents the overlay of two fluorescence signals.

### Example 10. In Vivo Rabbit Atherosclerotic Plaque Photodynamic Therapy Effect of LAM-Ce6 Nanoparticles

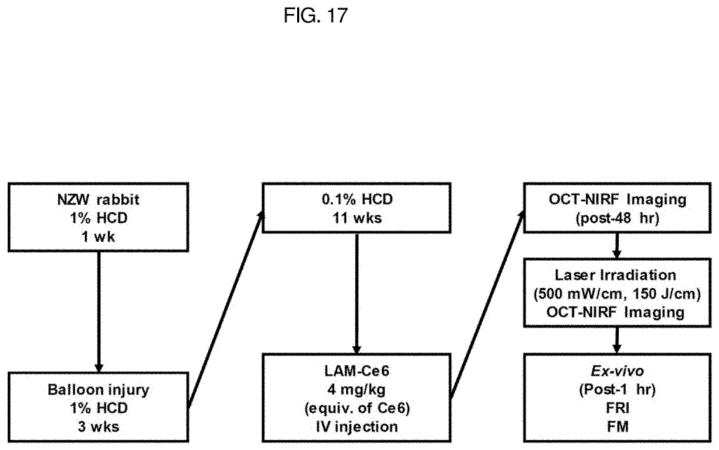

To confirm the photodynamic therapy (PDT) effect of LAM-Ce6 nanoparticles of the present disclosure on atherosclerotic plaques in a rabbit model, experiments were performed as in the protocol shown in FIG. 17.

In Vivo Image Analysis of Photodynamic Therapy Effects

Similar to the protocol shown in FIG. 17, a 1% cholesterol diet (HCD) was fed to a NZW rabbit for 1 week, and then the aortas were subjected to balloon injury. Then, a 1% cholesterol diet was maintained for 3 weeks, and then a 0.1% cholesterol diet was fed for 11 weeks, thereby generating atherosclerotic plaques. Atherosclerotic plaques in rabbits were imaged through OCT-NIRF at atherosclerotic plaques 48 hours after injection of LAM-Ce6 nanoparticles (4 mg/kg) (equivalent to Ce6) into the ear vein. To proceed with photodynamic therapy, a laser diffuser (670 nm, 500 mW/cm, 150 J/cm) was put into the aorta and located on the atherosclerotic plaque, and then the laser was irradiated. After irradiation, imaging was performed through OCT-NIRF.

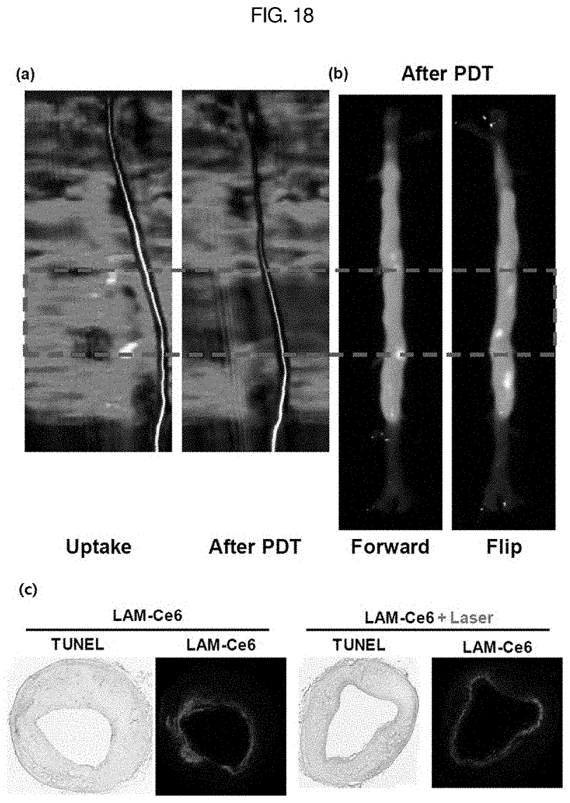

The imaging results are shown in FIG. 18(a) and FIG. 18(b).

As shown in FIG. 18(a) and FIG. 18(b), only in the laser-irradiated area, the NIRF signal decreased after laser irradiation as compared to before (baseline) laser irradiation. From the above results, the effect of photodynamic therapy that reduces the size of the arteriosclerotic plaque was confirmed.

OCT-NIRF In Vivo Fluorescence Imaging Method

Further, similar to the schematic diagram shown in FIG. 15, molecular imaging was performed through the OCT-NIRF system. Briefly, in the imaging system, when the light from the OCT laser light source was transmitted to the lens of the catheter through the core of the optical fiber, the signal reflected from the blood vessel was converted into an electrical signal, so that the shape of the blood vessel could be confirmed. In the NIRF system, when laser with 660 nm wavelength was transferred to a catheter through the core of the optical fiber, the LAM-Ce6 nanoparticles were photoactivated, and the emitted fluorescence was received back into the catheter, and then converted into an electrical signal, thereby confirming the intensity of the fluorescence.

Histological Analysis of Phototherapy Effect

To histologically analyze the effect of phototherapy, one hour after laser irradiation, the extracted carotid artery was sectioned to 10 μm, and the fluorescence image was confirmed. To confirm apoptosis by singlet oxygen, TUNEL (Terminal deoxynucleotidyl transferase dUTP nick end labeling) staining was performed. TUNEL is a staining method for confirming apoptosis that was used to detect DNA breakage formed when DNA fragmentation occurred in the final stage of apoptosis, and was obtained from Merck.

The results are shown in FIG. 18(c).

As shown in FIG. 18(c), LAM-Ce6 nanoparticles treatment and light treatment (LAM-Ce6+Laser) reduced the fluorescence signal in some areas as compared to controls treated with LAM-Ce6 nanoparticles alone, and TUNEL staining was also increased as compared to a control group.

Overall, from the above results, the inventors confirmed that photoactivation of LAM-Ce6 nanoparticles in atherosclerotic plaques in rabbits can induce apoptosis.

In Vivo Imaging and Histological Analysis of Atherosclerotic Plaque Retraction and Stabilization Through Phototherapy

The RAM11 PSR and ORO stainings were performed for histological analysis of atherosclerotic plaque retraction and stabilization through phototherapy. The RAM11 is an antibody capable of labeling rabbit macrophages, and was obtained from Dako. The PSR (Picro-Sirius Red) is a dye that can stain collagen, and was obtained from ScyTek (Picro-Sirius Red Stain Kit (For Collagen), PSR-1, ScyTek).

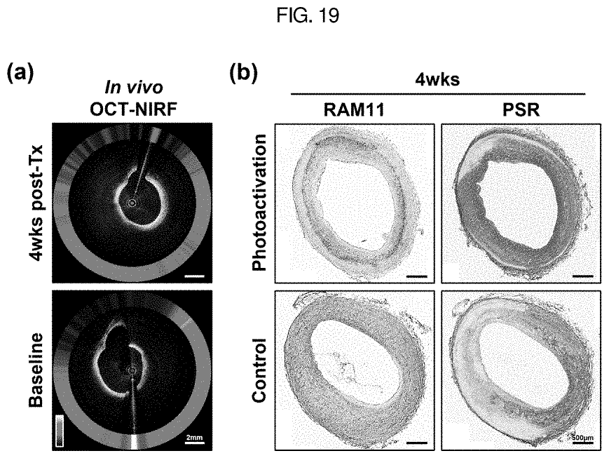

The results are shown in FIG. 19.

As shown in FIG. 19 (a), it was confirmed that the inflammatory signal was greatly reduced after 4 weeks of phototherapy in the area where the inflammatory signal was strong. As shown in FIG. 19(b), when compared with the control group not irradiated with the laser, it was confirmed that the macrophage and the size of atherosclerotic plaques was reduced through the decrease in the expression of RAM11. It was confirmed through PSR staining that the amount of collagen was increased compared to the control group.

Overall, from the above results, the inventors confirmed that photoactivation of LAM-Ce6 nanoparticles reduces the number of macrophages in rabbit arteriosclerotic plaques, allowing the atherosclerotic plaques to regress, and the increase in collagen can convert the atherosclerotic plaques into a good stable state.

### CONCLUSION

The present disclosure provides novel photoactivated theranostic nanoparticles that specifically bind to macrophage Dectin-1, for example, LAM-Ce6 nanoparticles according to an embodiment of the present disclosure. LAM-Ce6 nanoparticles exhibited negligible toxicity in the absence of light, and caused a large amount of macrophage apoptosis upon laser irradiation on macrophages. In addition, LAM-Ce6 nanoparticles were evaluated to have no toxicity in blood chemistry analysis and skin phototoxicity analysis. Moreover, the photoactivated theranostic nanoparticles of the present disclosure enable in vivo imaging of atherosclerotic plaque macrophages.

Therefore, when using the composition comprising, as an active ingredient, the nanoparticles comprising the near-infrared reactive photosensitizer covalently bonded to laminarin according to the present disclosure, it specifically binds to Dectin-1 of macrophages in atherosclerotic plaques and can be imaged by generating a near-infrared fluorescence signal only when irradiated with a near-infrared laser, and thus can be useful for imaging diagnostics. Further, it can induce apoptosis of atherosclerotic macrophages to stabilize the atherosclerotic plaque, reduce its size and reduce the macrophage-mediated inflammatory response, which can be usefully used for the prevention or treatment of arteriosclerosis.

The photoactivatable LAM-Ce6 nanoparticle fusion material of the present disclosure can targeting Dectin-1 present disclosure is expected to be promisingly used for selective fluorescence imaging and PDT in terms of stabilizing arteriosclerotic plaques.

What is claimed is:

1. Nanoparticles comprising a conjugate of laminarin and a near-infrared responsive photosensitizer covalently bonded thereto.

2. The nanoparticles according to claim 1, wherein the laminarin is located outside the nanoparticles and the near-infrared photosensitizer is located inside the nanoparticles.

3. The nanoparticles according to claim 1, wherein the nanoparticles are produced by self-assembly of conjugates.

4. The nanoparticles according to claim 1, wherein the near-infrared responsive photosensitizer is at least one selected from the group consisting of chlorin e6 (Ce6), porphyrin, photofrin, temoporfin, aminolevulinic acid-induced protoporphyrin IX, motexafin lutetium, padoporfin, padeliporfin, talaporfin (NPe₆), radachlorin, Purlytin, phthalocyanines, Verteporfin, HPPH (photochlor), TPC (5-(4-carboxyphenyl)-10,15,20-triphenyl-2,3-dihydroxychlorin), Chlorin p6 (Cp6), Purpurin-18, purpurinimide, and bacteriochlorin.

5. The nanoparticles according to claim 1, wherein the covalent bond is formed between a hydroxyl group of laminarin and a carboxyl group of the photosensitizer.

6. The nanoparticles according to claim 1, wherein the laminarin has an average molecular weight of 1 kDa to 9 kDa.

7. The nanoparticles according to claim 1, wherein the laminarin is contained in an amount of 60 wt % to 95 wt % based on the total weight of the nanoparticle.

8. The nanoparticles according to claim 1, wherein the near infrared responsive photosensitizer is contained in an amount of 5 wt % to 40 wt % based on the total weight of the nanoparticles.

9. The nanoparticles according to claim 1, wherein the nanoparticles have a diameter of 10 to 500 nm.

10. A composition comprising the nanoparticles of claim 1.

11. A method of preventing, diagnosing or treating arteriosclerosis comprising administering to a subject a composition comprising the nanoparticles of claim 1 as an active ingredient.

12. The method according to claim 11, wherein the method of preventing or treating arteriosclerosis further comprises irradiating a near-infrared light after administration of the composition to the subject.

13. The method according to claim 11, wherein the method for diagnosing arteriosclerosis further comprises,
irradiating a near-infrared light after administration of the composition to the subject; and
measuring the intensity of the near-infrared fluorescence signal emitted from the nanoparticles and comparing it with the result of a normal subject or an arteriosclerosis patient.
